(19) United States
(12) Patent Application Publication
(10) Pub. No.: US 2026/0065474 A1
(43) Pub. Date: Mar. 5, 2026

(54) DIAGNOSIS AND LOCALIZATION OF DISEASE STATES WITH AN ENSEMBLE OF FEATURE-FUSED TOP-PERFORMING QUANTITATIVE PARAMETER ARRAYS

(71) Applicant: Bernd Rolauffs, Emmendingen (DE)

(72) Inventor: Bernd Rolauffs, Emmendingen (DE)

(21) Appl. No.: 19/373,154

(22) Filed: Oct. 29, 2025

Related U.S. Application Data
(63) Continuation-in-part of application No. PCT/IB24/00210, filed on May 2, 2024.
(60) Provisional application No. 63/464,058, filed on May 4, 2023.

(51) Int. Cl. G06T 7/00; G06T 7/12; G16H 30/40; G16H 50/20

(52) U.S. Cl. CPC G06T 7/0012; G06T 7/12; G16H 30/40; G16H 50/20; G06T 2207/10056; G06T 2207/30024

(57) ABSTRACT

A universal biomarker array enables the diagnosis and localization of earlier-than-now recognizable disease states as well as increasing the accuracy of diagnosing states of disease(s) in general. The universal biomarker array is formed of a range of mathematical parameters computed from digital tissue images showing tissue details at a cellular level. The universal biomarker array includes one or more novel parameter arrays (namely, a spatial entropy array, a bin array, and/or a quartile array) in combination with one or more state-of-the-art parameter arrays (namely, a pattern array, a distance array, and/or a morphology array) and/or in combination with one or more other parameters. An AI/ML system can be used to analyze the universal biomarker array relative to database-based reference data for early diagnosis and localization of disease processes.

### CROSS-REFERENCE TO RELATED APPLICATION(S)

This patent application is a continuation-in-part (CIP) of International Patent Application No. PCT/IB2024/000210 entitled DIAGNOSIS AND LOCALIZATION OF DISEASE STATES WITH AN ENSEMBLE OF FEATURE-FUSED TOP-PERFORMING QUANTITATIVE PARAMETER ARRAYS filed May 2, 2024 and published as WO 2024/228056 on Nov. 7, 2024, which claims the benefit of U.S. Provisional Patent Application No. 63/464,058 entitled UNIVERSAL BIOMARKER ARRAY filed May 4, 2023, each of which is hereby incorporated herein by reference in its entirety.

### FIELD OF THE INVENTION

The invention generally relates to a universal biomarker array used across types of tissues and diseases in conjunction with AI/machine learning/statistical analysis for early disease diagnosis, localization of disease processes, and real-time feedback, usable during clinical and scientific imaging and image analysis.

### BACKGROUND OF THE INVENTION

In clinical medicine and basic science, a relevant focus is to improve methods for the diagnosis of earlier disease processes (earlier diagnosis) of many diseases, a more accurate diagnosis and the precise localization of early disease stages or disease in general within tissues, preferably combined with near-instant feedback.

Currently, depending on the type of tissue and disease, a range of specialized state-of-the-art diagnostic methods exists for non-clinical use and for clinical use, e.g., laboratory (blood, urine tests), functional, and genetic tests, clinical imaging (magnetic resonance imaging (MRI), CT scan), endoscopy, and harvesting biopsies, e.g., for histology or RNA-Seq. Non-clinical tests, e.g., basic science biochemical assays, can determine and localize early disease processes well but often require tissue biopsies and tissue-destructive analyses, which is not possible or not ethically justifiable for all tissues. Clinical tests, e.g., genetic, laboratory and functional tests do not allow localization of disease processes; they determine the risk for a certain disease, the systemic presence of a disease-specific biomarker, or the degree of limitation through disease. Liquid biopsies are also location-unspecific. In contrast, imaging tests are important for localizing diseases, but they are limited by their technical resolution. In some research studies, MRI scanners achieved spatial resolutions of less than 0.5 mm, whereas clinical MRI scanners achieve a spatial resolution of approximately 1-2 mm in routine imaging studies but generally do not reach the resolution to clinically visualize early disease processes, e.g., on the microscopic scale. Thus, MRI is not sensitive enough to detect small changes in tissue architecture in early disease states. Modern endoscopy, e.g., confocal micro-endoscopy, can have microscopic resolution and could be useful for identifying early-stage disease in near-real time using the disease-specific markers and downstream methodology described herein such as to accurately diagnoses early disease. Other uses of images of organs/tissues/tissue section(s) for medical diagnosis include the assessment by a pathologist or other specialist relying on professional experience or by an AI/machine learning system relying on state-of-the-art parameters.

### SUMMARY OF VARIOUS EMBODIMENTS

In accordance with one embodiment of the invention, a method and a system for disease diagnosis comprises receiving digital tissue images showing tissue details at a cellular level; performing automated image analysis on the received digital tissue images to identify cells of interest for analysis; calculating a range of mathematical parameters on multiple levels for collective use as a universal biomarker array including a pattern array, a distance array, a morphology array, a spatial entropy array, a bin array in which absolute parameter values are translated to relative information by assigning them to specific bin positions, and a quartile array in which absolute parameter values are translated to relative information by assigning values to specific quartiles; and analyzing the universal biomarker array relative to database-based reference data for early diagnosis and localization of disease processes.

In various alternative embodiments, the digital tissue images may include endo-microscopy digital tissue images. Performing automated image processing may involve identifying cells of interest relative to an image background, building at least one region of interest (ROI) in a non-background region, and performing ROI-based segmentation (i) of cells for analyses of the segmented cells and (ii) analyses of an inverted segmentation for analyses of spaces between the cells. The array of spatial entropy parameters may include Batty (absolute, relative), Contagion, Karlstrom (absolute, relative), O Neill (absolute, relative), and Parredw parameters. Calculating the bin array may involve assigning each of a plurality of parameters into relative class-and range-specific bin positions across an entire range of parameter values, wherein a total number of bins is calculated by multiplying a number of disease states with a number of bins per state, and wherein the translation of data into relative class-and range-specific bin positions may be performed on the parameters of the spatial entropy array, the pattern array, the distance array, and the morphology array. Calculating the quartile array may involve assigning each of a plurality of parameter values to a disease state-specific quartile position and translating each of the plurality of parameter values into a disease range-specific quartile position, wherein the translation of data into relative class-and range-specific quartile positions may be performed on the parameters of the spatial entropy array, the pattern array, the distance array, and the morphology array. Analyzing the universal biomarker array relative to database-based reference data may involve providing the universal biomarker array to an AI/ML system trained on universal biomarker array data to detect early diagnosis and localization of disease processes, wherein the AI/ML system may utilize random forest regression or other tree-based classification/regression to detect early diagnosis and localization of disease processes.

Additional embodiments may be disclosed and claimed.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 shows a random forest classification using the universal biomarker array for diagnosing KINs vs. SCC in accordance with certain embodiments.

FIG. 2 shows a predictive classification using the universal biomarker array for diagnosing colon adenoma vs. cancer in accordance with certain embodiments.

FIG. 3 is a graph showing significantly increased classification accuracies of human articular cartilage disease states, resulting from using novel parameter arrays (n=3) and novel unique array combinations as distinct predictive modeling input datasets (n=57), compared to state-of-the-art parameters (n=3).

FIG. 4 is a graph showing significantly increased classification accuracies of human skin disease states, resulting from using novel parameter arrays (n=3) and novel unique array combinations as distinct predictive modeling input datasets (n=57), compared to state-of-the-art parameters (n=3).

FIG. 5 is a graph showing significantly increased classification accuracies of human colon disease states, resulting from using novel parameter arrays (n=3) and novel unique array combinations as distinct predictive modeling input datasets (n=57), compared to state-of-the-art parameters (n=3).

FIG. 6 is a graph showing significantly increased classification accuracies of a broad range of unique array combinations, serving as distinct predictive modeling input datasets in a complex environment generated by pooling all tissues/disease states.

FIG. 7 is a graph showing state-of-the-art and novel single arrays and top unique array combinations that consistently achieved the highest classification accuracies in all analyzed tissues/disease states.

FIG. 8 is a graph showing the feature-fused ensemble of top-performing unique array combinations and their average disease state classification accuracy performance across all tissues/disease states relative to state-of-the-art parameters.

FIG. 9 is a schematic diagram showing six constituent arrays of a universal biomarker array in accordance with certain embodiments.

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Definitions. As used in this description and the accompanying claims, the following terms shall have the meanings indicated, unless the context otherwise requires.

A “set” includes one or more members, even if the set description is presented in the plural (e.g., a set of Xs can include one or more X).

The concept of “real-time” can range from instant to seconds or minutes depending on the context. For example, real-time feedback for a medical procedure generally would require that the feedback be provided during the course of the medical procedure and in some cases can require that the feedback be provided within a required timeframe (e.g., if tissue damage would occur within X minutes of detecting the start of a disease state/process such as loss of oxygen, then real-time feedback would need to be provided within X minutes for it to be useful in taking remedial action).

The term “disease” as in a disease state, process, diagnosis, etc. is used herein generically to refer to an attribute or combination of attributes that is indicative of some distinguishing condition, which could be a disease, injury, defect, allergic or chemical/drug reaction, hyperactivity, hypoactivity, healing, regeneration, growth, or other condition that can be analyzed using the techniques described herein.

In view of the background above, there is a clear need for improving the sensitivity, accuracy, localization, and speed of feedback of methods that detect/diagnose and localize earlier disease processes. Three short examples highlight the advantages of a methodology capable of fulfilling these above stated points:
Earlier detection of cancer: A methodology capable of detecting the earlier stages of certain skin or other tumors non-invasively and in near-real time would become instantaneously clinically relevant, as earlier therapy would be in reach,
Earlier detection of tissue damage/degeneration: With current clinical technology, we are unable to pinpoint early tissue damage/degeneration, which are the early, potentially reversible disease states of osteoarthritis (OA), a degenerative joint disease that remains as the leading cause of global disability. Consequently, we cannot test novel/promising drug candidates early enough in the potentially therapy-susceptible early disease states, thereby missing the windows of opportunities. A methodology capable of detecting earlier stages of OA (or other diseases) would allow developing novel, disease-state-specific drugs and/or testing existing, promising drugs that failed in clinical trials because they were tested too late in the process of disease,
Real-time feedback: Feedback on whether a given tissue area is or is not disease-affected would improve the local precision of diagnostic biopsies (skin, intestine), tumor resection (resection margin), and the precise implantation of regenerative medicine implants during surgical procedures (therapy of cartilage defects).

Collectively, the capabilities of earlier, more accurate diagnosis and precise location paired with real-time feedback would significantly improve developing treatment options and treatment outcomes and would generate the chance to improve meaningfully medical treatment pathways.

To enable the diagnosis and localization of earlier-than-now recognizable disease states and to increase the accuracy of diagnosing states of disease(s) in general, the inventor has developed an ensemble of top-performing quantitative parameter arrays by feature fusion. Specifically, certain embodiments calculate, from images of organs, tissues, or tissue sections (referred to herein generically as “tissue”) showing details at a cellular level, various mathematical parameters (e.g., spatial entropy, cell morphology, distribution and density, and population-based parameters such as described/defined below) that are collectively used across types of tissues and diseases for quantitatively describing healthy vs. specific disease states of organs/tissues or tissue sections. For convenience, sets of related mathematical parameters may be logically categorized into individual arrays (where each individual array essentially translates distinct facets of tissue architecture in health and changes of tissue architecture in disease(s) into comprehensive quantitative parameters based on images taken with cell-depicting resolution/methodology), and multiple arrays may be analyzed collectively as a feature-fused ensemble (referred to herein as a “universal biomarker array”). It should be noted that the term “universal” here reflects that the biomarker array applies to a wide range of organs/tissues or tissue sections and/or healthy vs. disease states, and various embodiments may use different arrays and/or arrays having different constituent mathematical parameters as a universal biomarker array such as for different organs/tissues and/or for detecting different healthy vs. disease states. The universal biomarker arrays may be provided as predictive modeling input data to an artificial intelligence/machine learning system such as for classifying healthy vs. disease states or for basic statistics.

Among other things, such mathematical biomarker arrays and associated methodologies can be used for such things as:
differentiating healthy vs. early disease states (early disease diagnosis) in an organ/tissue or tissue section
localization of disease processes in an organ/tissue or tissue section
real-time feedback on disease state and localization during imaging (decision-support tool e.g., during surgical procedures) in an organ/tissue
monitoring of disease processes and tissue structures (e.g., healing/regeneration, drug treatment results, cosmetic application results, etc.)
diagnosis and staging of tumors
improving the diagnosis of disease states in an organ/tissue or tissue section in general, compared to state-of-the-art, by increased accuracy for example, by statistical and/or artificial intelligence (AI)/machine learning (ML) supported testing of images/data of interest against healthy and/or disease state-specific database-based reference data. It should be noted that, while “early stage” detection is often mentioned herein, the techniques described herein can provide benefits in other applications. For example, later stage disease diagnosis may benefit from this universal biomarker array because it may be better than current methods.

Certain embodiments innovatively describe tissue architecture in health and early disease quantitatively with mathematical parameters (termed universal biomarker array) that describe cell population characteristics and matrix characteristics, which are used as AI/ML input or other statistical analyses for diagnosis and can be used whenever imaging/images allow identification/segmentation of cells (and matrix) within organs/tissues/tissue section(s) and imaging/images of cells.

Generally speaking, a given tissue's cell population characteristics are remarkably tissue type-specific and also disease-sensitive, e.g., in trauma cells are lost, in tissue swelling the cells are spaced farther apart, and in cancer and other proliferative diseases cell numbers increase, cells are very differently placed or different types of cells are present, compared to healthy tissues. Thus, mathematical parameters that quantify a range of characteristics of a tissue's cell population(s) constitute together a disease-sensitive digital fingerprint of tissue architecture and function. Similarly, a given tissue's extracellular matrix—the space between cells—can undergo various changes in early disease, which can be described with mathematical parameters in health and early disease. These data are used as AI input for early diagnosis and disease localization across tissue types and diseases, and constitute, thus, a diagnostically usable universal biomarker array.

The here-used AI/ML input is different from other diagnostic AI/ML applications that use as input (i) annotated medical images without further quantification, e.g., for convolutional neural network analysis, or (ii) that use simpler quantifications, e.g., Haralick texture features, of segmented images, or (iii) in which the segmentation does not identify cell populations/matrix, e.g., because images from MRI (or other imaging modalities) were used whose resolution is too low for analyzing cells/matrix.

The following is a description of a workflow in accordance with certain embodiments.

The workflow generally begins by generating images of organs/tissues/tissue sections (e.g., with endo-microscopy, microscopy, or other imaging technologies) and/or using existing images of organs/tissues/tissue sections (e.g., from histological sections generated for assessment by the pathologist or other specialists). Importantly, as discussed above, embodiments of the present invention operate on images with a resolution that depicts cells. A specific method for generating images for the here described analysis is to use endo-microscopy that is commercially available for clinical use, e.g., from the companies Mauna Kea Technologies (e.g., Cellvizio, a probe-based confocal laser endo-microscopy system) or from Zeiss (e.g., CONVIVO, a confocal endo-microscopy system), or other suitable systems for clinical use. A specific method for using images from histological sections for the here described analysis is to use digital pathology slide scanners for generating images or using existing scans. A specific method for generating non-clinical images from organs/tissues/tissue sections for the here described analysis is to use any type of microscopy, e.g., fluorescent, confocal, or other microscopy techniques. If images are generated for clinical use, e.g., using endo-microscopy, certain embodiments may use auto-fluorescent drugs, e.g., antibiotics such as Tetracycline or Ciprofloxacin, or Chinin as fluorescent dye, or fluorescent molecules (i.e., where such molecule is not a drug in a pharmacological sense), as currently no clinically usable fluorescent cell dye is available. It should be noted that the drugs (e.g., antibiotics) are not being used here for their pharmacological activity. This is not needed for all but some tissues, e.g., articular cartilage, in which cells are not readily recognizable. If auto-fluorescent drugs do not readily penetrate the tissue(s), e.g., cartilage, drug polarization is carried out, e.g., by adjusting the pH/using another charged/polar molecule in solution. For example, Tetracycline penetration into negatively charged tissues, for using Tetracycline as fluorescent dye for visualizing the tissue's cells, e.g., in cartilage, can be achieved by adding a positive charge to the solution to interact with the negatively charged functional groups of Tetracycline. This can be achieved with polar solvents such as PEG and glucose solutions, e.g., 70% or 20%. Specifically, Tetracycline solution for clinical use (e.g., Doxycycline vials for i.v. usage) can be used in conjunction with around 20%-70% glucose solution for clinical use. If needed, tissue penetration of ciprofloxacin and other auto-fluorescent polar drugs can be achieved by similar means. Since doxycycline can interact with divalent cations such as magnesium and calcium leading to increased fluorescence, divalent cations can be utilized in conjunction with doxycycline for imaging by adding, e.g., magnesium ions, or by using isotonic solutions commonly used for arthroscopic surgery that contain divalent cations, e.g., magnesium ions. In this context, the fluorescent signal can be excited in the range of 350-400 nm, e.g., with UV-A light. Another possibility for using an auto-fluorescent drug as fluorescent dye for visualizing the tissue's cells is to synthesize a drug derivative that has a positively charged group, e.g., an amino group, attached to it. It should be noted that other imaging technologies, including future-developed imaging technologies or even enhanced/improved versions of current imaging technologies that do not have a cellular resolution (e.g., existing MRI and CT imaging technologies), could be used in alternative embodiments of the present invention.

The workflow generally continues with automated image analysis for recognizing in images organs/tissues/tissue sections/cells vs. image background, building regions of interest (ROIs) in non-background regions, and ROI-based segmentation (i) of cells for analyses of the segmented cells and (ii) analyses of the inverted segmentation for analyses of the space between the cells (the matrix).

The workflow generally continues with calculation of a range of mathematical parameters on multiple levels that are collectively used as a universal biomarker array and that are described/defined below.

The workflow generally continues with storing curated (e.g., organ/tissue/tissue section/cell-and disease state-specific) diagnosed/classified/annotated universal biomarker array data in a database to produce database-based reference data.

The workflow generally continues with using images/data of interest for statistical and/or AI/ML supported testing against database-based reference data for early diagnosis and localization of disease processes. One specific AI method is to use random forest classification for diagnostic purposes, e.g., to test whether (i) “new” data from an image of interest represents a healthy state or a diseased state, and/or (ii) determines a specific disease classification/score. Here, “new” data from “new” images of interest are tested against reference data, which may be stored in a database or otherwise available to the system. Another specific AI method is to use tree-based classification/regression such as random forest regression for calculating a continuous numerical value, e.g., a continuous score or other values of interest, in which “new” data from “new” images of interest are tested against database reference data. Another specific AI method is to use LightGBM classification as described for random forest classification. Another specific AI method is to use PLS-DA for discriminant analysis (e.g., with max.dist, centroids.dist and/or mahalanobis.dist) and/or sPLS-DA for selecting a subset of variables for discriminant analysis and prediction/classification of “new” data. Generally, accuracy, precision, recall, and F1-score of each model are used to decide on the specific AI model.

As a result, the workflow determines whether images/data of interest represent healthy organ/tissue/tissue section data or depart from healthy data, e.g., a specific disease state can be diagnosed and localized and can also determine the accuracy (classification) of diagnosis and other quality control-related parameters. Also, because embodiments allow for analysis of tissues and tissue sections, the universal biomarker array is applicable to cells within tissues and cells visible in tissue sections. Thus, for example, universal biomarker arrays may be particularly applicable in pathological institutes (tissue sections) and in clinical trials/routine (patients/live cell imaging). Also, because embodiments allow for analysis using different staining solutions (dyes) but also for analysis using live cell imaging, universal biomarker arrays of the present invention are dye-independent.

The following is a description of various mathematical parameters that are currently contemplated for universal biomarker arrays of certain embodiments, which may be used as AI/ML input or other statistical analyses for early diagnosis and disease localization across tissue types and diseases:
calculated with the “R” language and the library “SpatEntropy” from point patterns created from cell coordinates:
   Spatial entropy Batty (batty)
   Spatial entropy Contagion (contagion)
   Spatial entropy Karlstrom (karlstrom)
   Spatial entropy O'Neill's (oneill)
   Spatial entropy Parredw (parredw)
   Spatial entropy Leibovici (leibovici)
   Spatial entropy Altieri (altieri),
   using absolute and relative values when available
calculated with the “R” language and the library “SpatEntropy” from marked point patterns created from cell coordinates, using all parameters calculated for each cell as marks:
   Shannon's spatial entropy (shannon), using absolute and relative values
calculated with the “R”language and the library “spatstat”:
   for each cell:
      nearest neighbor distance (nndist)
   for each image ROI/image from point patterns created from cell coordinates:
      mean and average nndist
      1. Quartile of nndist
      2. Quartile of nndist
      3. Quartile of nndist
      Standard deviation of nndist
      Standard error of nndist
      Spatial Intensity
      Clark Evans Aggregation Index (clarkevans) calculated with no edge corrections, “cdf” edge corrections, and “Donnelly” edge corrections
      mean and average of the Clark Evans Aggregation Index versions
      pair correlation function maximum (maximum of pcf calculated with “trans” and “iso” corrections)
      r-value (x-axis) of pair correlation function maximum on y-axis (calculated with both Ohser-Stoyan translation and Ripley's isotropic correction)
calculated with ImageJ/Fiji, or “R” language:
   for each cell:
      length
      width
      aspect ratio
      roundness
      circularity
      solidity
      perimeter
      area
      orientation
for each image ROI/image parameter, if applicable:
   mean, median, standard deviation, standard error, quartiles

The above listed parameters focus on a given tissue's cell population and are calculated using cell identification/segmentation. Inversion of the segmentation mask selects the extracellular matrix, i.e., the space between cells within a tissue. From the segmentation/areal data, quantitative parameters such as spatial entropy parameters (see above for parameters; library “SpatEntropy”) and Haralick texture features can be calculated.

Certain embodiments introduce three novel quantitative parameter arrays, as follows:
(a) A novel quantitative parameter array termed spatial entropy (abbreviated entropy), for which various spatial entropy parameters are calculated from cell positions (details below).
(b) A novel quantitative parameter array termed bin, in which absolute parameter values are translated to relative information by assigning them to specific bin positions. Binning refers to the process of dividing a continuous parameter data set into consecutively numbered intervals (bins) and assigning each data point to a specific bin number, based on its value. Specifically, each parameter value is translated into a disease state-specific bin position, determined separately for each disease state using 20 bins per disease state. Additionally, each parameter value is translated into a disease range-specific bin position. Here, bin positions across the entire range of parameter values are assigned and the total number of bins is calculated by multiplying the number of disease states with the number of bins per state, e.g., 20 bins. The translation of data into relative class-and range-specific bin positions is so far performed on the novel entropy array and on the state-of-the-art morphology, pattern, and distance arrays but will in the future not necessarily be limited to those.
(c) A novel quantitative parameter array termed quartile, in which absolute parameter values are translated to relative information by assigning values to specific quartiles. Specifically, each parameter is assigned to a disease state-specific quartile position and calculations are performed independently for each state. Additionally, each parameter value is translated into a disease range-specific quartile position, in which the entire range of values is used for quartile calculations and assigning each value to these quartiles. The translation of data into relative class-and range-specific quartile positions is so far performed on the entropy, morphology, pattern, and distance arrays but will in the future not necessarily be limited to those.

In one exemplary embodiment, the entropy array includes the spatial entropy parameters Batty (absolute, relative), Contagion, Karlstrom (absolute, relative), O Neill (absolute, relative), and Parredw, which may be calculated with the R package ‘SpatEntropy.’ It is important to note that the exact number and/or type of spatial entropy parameters that are currently contemplated for the entropy array are not to be understood an exclusive final list because the number and/or type of spatial entropy parameters could vary in different embodiments. For that matter, it is important to note that the exact number and/or type of parameters that are currently contemplated for any array are to be understood as an exclusive final list because the number and/or type of parameters could vary in different embodiments.

In certain embodiments, the entropy, bin, and quartile arrays are used in combination with one or more of the following additional arrays (sometimes referred to state-of-the-art arrays) as a feature-fused ensemble that can be used for predictive modeling:
(d) A pattern array that in one embodiment includes the parameters: spatial intensity, the Clark-Evans Index with no edge correction, the Clark-Evans Index with cdf edge correction, the Clark-Evans Index with Donnelly edge correction, the pair correlation function maximum, and the x value at which the maximum is observed (with both Ohser-Stoyan translation and Ripley's isotropic correction), which may be calculated with the R package ‘spatstat.’
(e) A distance array that in one embodiment utilizes the individual nearest neighbor distances that are determined for each cell and its nearest neighbor (e.g., using the spatstat package for R) and in one embodiment includes the calculated mean, median, and standard deviation as well as the first, second, and third quartiles.
(f) A morphology array that in one embodiment utilizes the individual cell morphologies that are determined for each cell and in one embodiment includes the calculated averages for cell area, length, width, orientation, perimeter, circularity, and solidity.

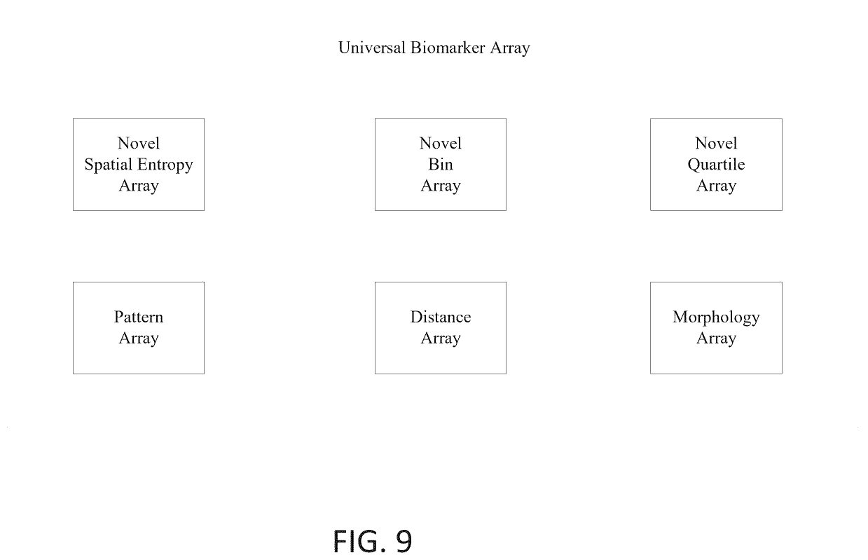

It should be noted that, while certain embodiments may use all six of these arrays and optionally also other parameters, as depicted schematically in FIG. 9, alternative embodiments can include one or more of the novel arrays in combination with one or more of the state-of-the-art arrays and/or in combination with one or more other parameters. Also, as discussed above, the constituent parameters of each array can differ between different embodiments.

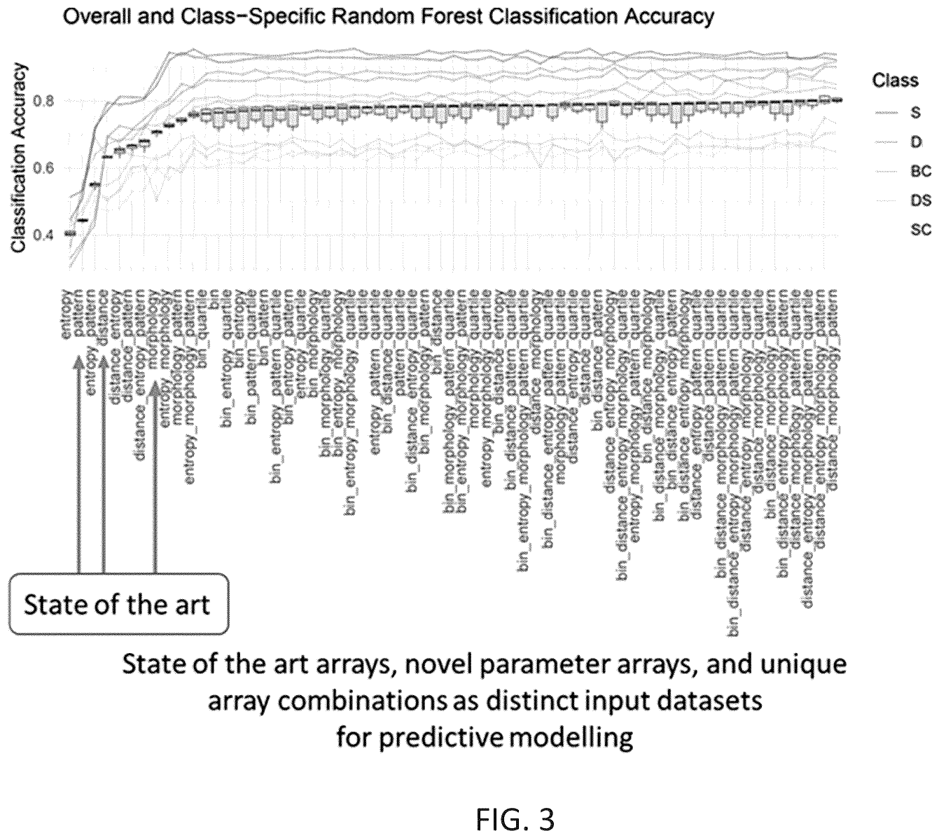

In order to generate distinct unique array combinations and to compare the disease state classification accuracies of single arrays vs. unique combinations of arrays vs. state-of-the-art parameters as predictive modeling inputs, the morphology, pattern, distance, entropy, bin, and quartile array values were calculated for a range of diseases/disease states in three human tissues: articular cartilage, skin, and colon (details below). The arrays were used as single arrays (6 in total; 3 are state-of-the-art: pattern, distance, morphology) and for generating unique array combinations (combinations of 2, 3, 4, 5, and 6 arrays; 57 unique combinations in total). This resulted in 63 different array/array combinations, which were used as distinct predictive modeling inputs for random forest modeling. The modeling process, carried out in “R,” included 5-fold cross validation and 5 times cross-validation, and was performed two times, using balanced class (disease state) distributions with an equal number of data rows per class for all runs. This step allowed analyzing the resulting accuracies as a function of the 63 distinct predictive modeling inputs (FIGS. 3-5), which was performed to identify those single arrays and unique combinations of arrays that had significantly higher classification accuracies than state-of-the-art parameters. Statistical analyses were performed on three data subsets. Each included all novel arrays, all novel unique array combinations, and one of the three state-of-the-art parameter sets, as indicated in detail in the figure legends for FIGS. 3-5.

In human articular cartilage (FIG. 3), statistical tests revealed that the disease state classification accuracies of all novel arrays and all unique array combinations were significantly different from the accuracies that resulted from using the state-of-the-art parameter arrays: pattern (p<0.001), distance (p<0.001), and morphology (p<0.001). As demonstrated by FIG. 3, using the novel array bin alone and using most unique array combinations as predictive modeling inputs (except entropy_pattern, distance_entropy, distance_pattern, distance_entropy_pattern array combinations) led, in articular cartilage, to significantly higher classification accuracies than using the state-of-the-art parameters, clearly surpassing the performance of state-of-the-art parameters.

As shown in FIG. 3, which is a graph showing significantly increased classification accuracies of human articular cartilage disease states, resulting from using novel parameter arrays (n=3) and novel unique array combinations as distinct predictive modeling input datasets (n=57), compared to state-of-the-art parameters (n=3), the box plots illustrate the overall accuracies that resulted from using distinct predictive modeling input datasets; they give the median and the 25th and 75th percentiles and the whiskers give the 10th and 90th percentiles. The line plots illustrate the specific accuracies that were calculated for each disease state (class), using the confusion matrix that resulted from modeling. The individual disease states describe how the cells were arranged in spatial patterns (e.g., in strings, double strings, small and big clusters, and in a diffuse arrangement without discernible spatial patterns), as such arrangements correlate with structural and functional pathology (details are described below). These disease state-specific accuracies indicate the accuracy of correctly classifying a particular disease state and indicate how well the model performs for individual disease states. The Shapiro-Wilk tests, which test for data normality, revealed that all datasets were non-normally distributed. The Wilcoxon rank-sum tests for pairwise comparisons with p-values adjusted using the Bonferroni method revealed that each of the state-of-the-art arrays pattern, distance, and morphology was significantly different (each p<0.0005) from each novel array and each unique array combination. Thus, significant differences are not indicated.

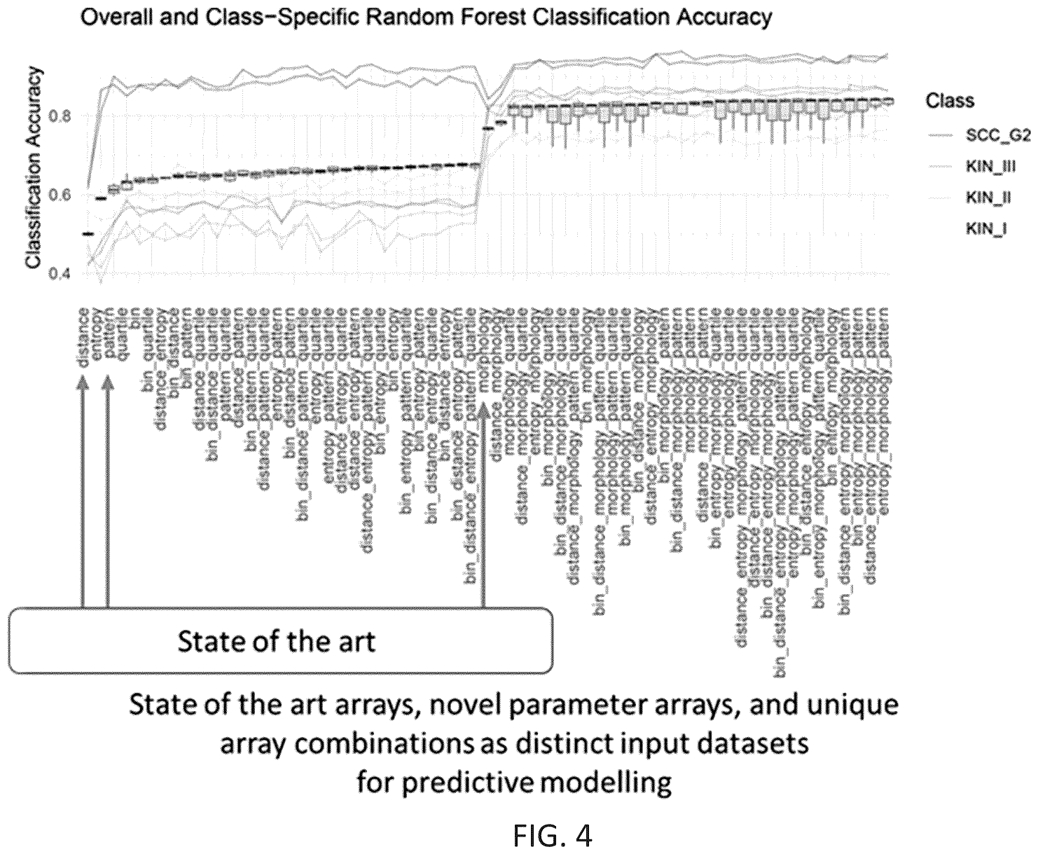

In the human skin (FIG. 4), statistical tests revealed that the disease state classification accuracies of all novel arrays and all unique array combinations were significantly different from the accuracies that resulted from using the state-of-the-art parameter arrays pattern (p<0.001), distance (p<0.001), and morphology (p<0.001) as predictive modeling inputs. As illustrated in FIG. 4, the distinct array combinations located to the right of the morphology array on the x-axis of FIG. 4 yielded significantly higher classification accuracies in human skin, compared to utilizing state-of-the-art parameters, thereby clearly surpassing the performance achieved with the current state-of-the-art parameters.

As shown in FIG. 4, which is a graph showing significantly increased classification accuracies of human skin disease states, resulting from using novel parameter arrays (n=3) and novel unique array combinations as distinct predictive modeling input datasets (n=57), compared to state-of-the-art parameters (n=3), the box plots illustrate the overall accuracies that resulted from using distinct predictive modeling input datasets; they give the median and the 25th and 75th percentiles and the whiskers give the 10th and 90th percentiles. The line plots illustrate the specific accuracies that were calculated for each disease state (class), using the confusion matrix that resulted from modeling. The individual disease states were actinic keratosis graded as KIN (keratinocyte intraepidermal neoplasia) I, II, or III, which are pre-malignant lesions, and a moderately differentiated squamous cell carcinoma (SCC G2), a malignant cancer (details are described below). These disease state-specific accuracies indicate the accuracy of correctly classifying a particular disease state and indicate how well the model performs for individual disease states. The Shapiro-Wilk tests, which test for data normality, revealed that all datasets were non-normally distributed. The Wilcoxon rank-sum tests for pairwise comparisons with p-values adjusted using the Bonferroni method revealed that all novel arrays were significantly different from each of the state-of-the-art arrays pattern, distance, and morphology (0.000001<p<0.007). Moreover, each unique array combination was significantly different (0.000001<p<0.007) from the state-of-the-art and the novel single arrays. Thus, significant differences are not indicated.

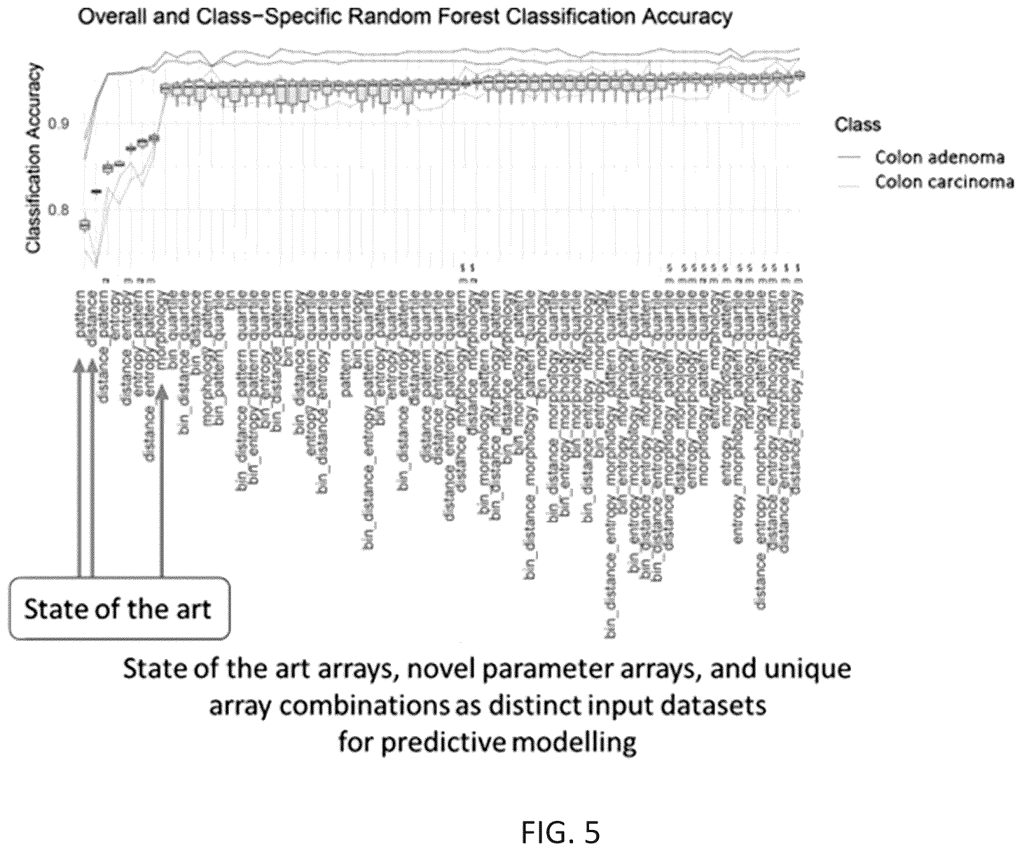

In the human colon (FIG. 5), statistical tests revealed multiple significant differences in the classification accuracies when using state-of-the-art vs. the novel array entropy and a range of unique array combinations, which are detailed in the figure legend. As demonstrated by FIG. 5, a range of unique array combinations (indicated with ‘§ ’) led in the human colon to significantly higher classification accuracies than using state-of-the-art parameters as predictive modeling inputs, clearly surpassing the performance of state-of-the-art parameters.

As shown in FIG. 5, which is a graph showing significantly increased classification accuracies of human colon disease states, resulting from using novel parameter arrays (n=3) and novel unique array combinations as distinct predictive modeling input datasets (n=57), compared to state-of-the-art parameters (n=3), the box plots illustrate the overall accuracies that resulted from using distinct predictive modeling input datasets; they give the median and the 25th and 75th percentiles and the whiskers give the 10th and 90th percentiles. The line plots illustrate the specific accuracies that were calculated for each disease state (class), using the confusion matrix that resulted from modeling. The individual disease states were colonic adenoma and colonic carcinoma (details are given in the text section Analyzed tissues, diseases, and disease states). These disease state-specific accuracies indicate the accuracy of correctly classifying a particular disease state and indicate how well the model performs for individual disease states. The Shapiro-Wilk tests, which test for data normality, revealed that all datasets were non-normally distributed. The Wilcoxon rank-sum tests for pairwise comparisons with p-values adjusted using the Bonferroni method revealed that the novel entropy array was significantly different from each of the state-of-the-art arrays pattern, distance, and morphology (each p<0.0001), whereas the novel bin and quartile arrays were not. Moreover, the state-of-the-art arrays pattern and distance were significantly different from each unique array combination (0.000001<p<0.02). The state-of-the-art array morphology was significantly different from a range of unique array combinations (0.000001<p<0.02) indicated with ‘m.’ Other significant differences are not indicated.

The symbol ‘§ ’ indicates unique array combinations, which, when used as predictive modeling inputs, led to significantly higher classification accuracies than any of the state-of-the-art parameters.

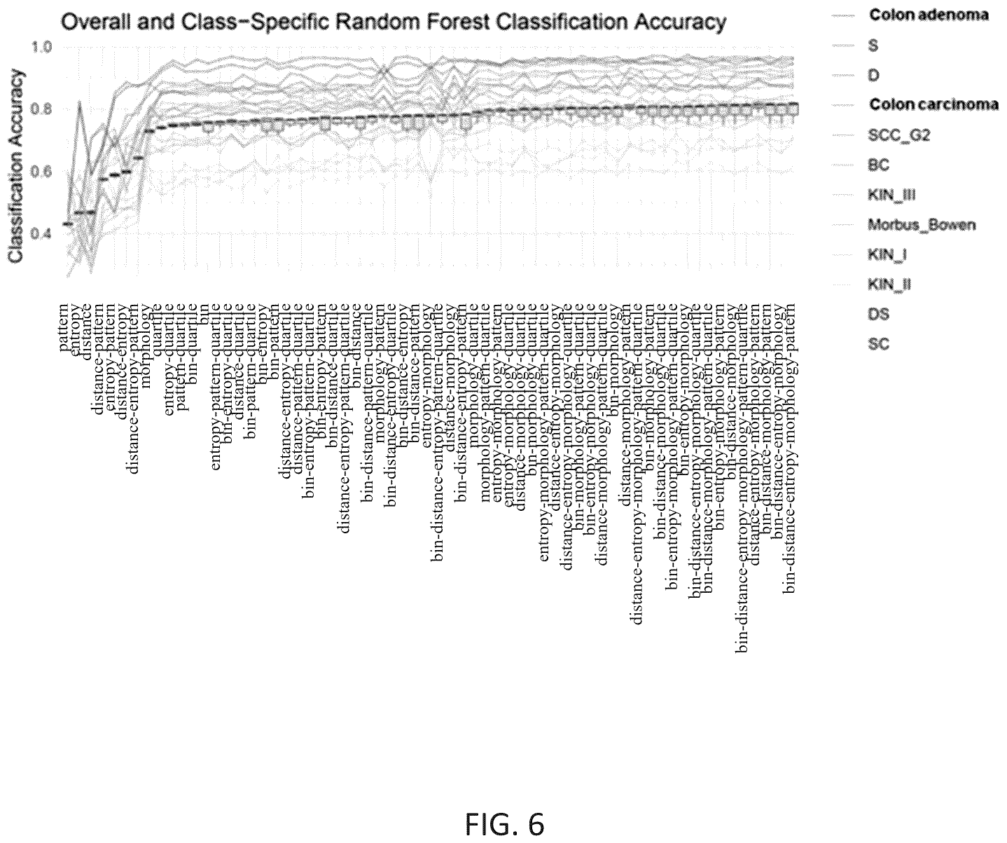

Next, the novel single arrays and the unique array combinations were tested as predictive modeling inputs in a complex environment against the state-of-the-art arrays by pooling all tissues/disease states, which generated 12 different classification choices. Using the pooled data (FIG. 6), statistical tests revealed that all disease state classification accuracies except the comparison between the arrays distance and entropy reached significance (0.0000001<p<0.03). Importantly, the novel array bin and all distinct array combinations located to the right of the morphology array on the x-axis of FIG. 6 yielded significantly higher classification accuracies than the state-of-the-art parameters. Note, this superior performance was attained in the context of cross-classification across diverse tissues and disease states, highlighting the efficacy of the novel, distinctive array combinations as predictive modeling inputs for classification tasks. This accomplishment serves as evidence of the practical utility that these unique array combinations bring to complex, real-world scenarios, affirming their potential as versatile tools for advanced classification applications. Thus, FIGS. 3-5 presented the disease state classification accuracies in specific tissues and FIG. 6 in a pooled, complex dataset. Together the figures demonstrated statistically and convincingly that a range of unique array combinations clearly surpass the accuracy resulting from state-of-the-art parameters when used as predictive modeling inputs.

As shown in FIG. 6, which is a graph showing significantly increased classification accuracies of a broad range of unique array combinations, serving as distinct predictive modeling input datasets in a complex environment generated by pooling all tissues/disease states, the individual tissues/disease states used in FIGS. 3-5 were pooled for generating a challenging classification task. The box plots (pink) illustrate the overall accuracies that resulted from using distinct predictive modeling input datasets; they give the median and the 25th and 75th percentiles and the whiskers give the 10th and 90th percentiles. The line plots illustrate the specific accuracies that were calculated for each disease state (class), using the confusion matrix that resulted from modeling. These disease state-specific accuracies indicate the accuracy of correctly classifying a particular disease state and indicate how well the model performs for individual disease states. The Shapiro-Wilk tests, which test for data normality, revealed that all datasets were non-normally distributed. The Wilcoxon rank-sum tests for pairwise comparisons with p-values adjusted using the Bonferroni method revealed that the disease state classification accuracies of all novel arrays and all unique array combinations were significantly different (0.0000001<p<0.03) from the accuracies that resulted from the art parameter arrays pattern, distance, and morphology, used as predictive modeling inputs. The only exemption was the comparison between the arrays distance and entropy, which did not reach significance.

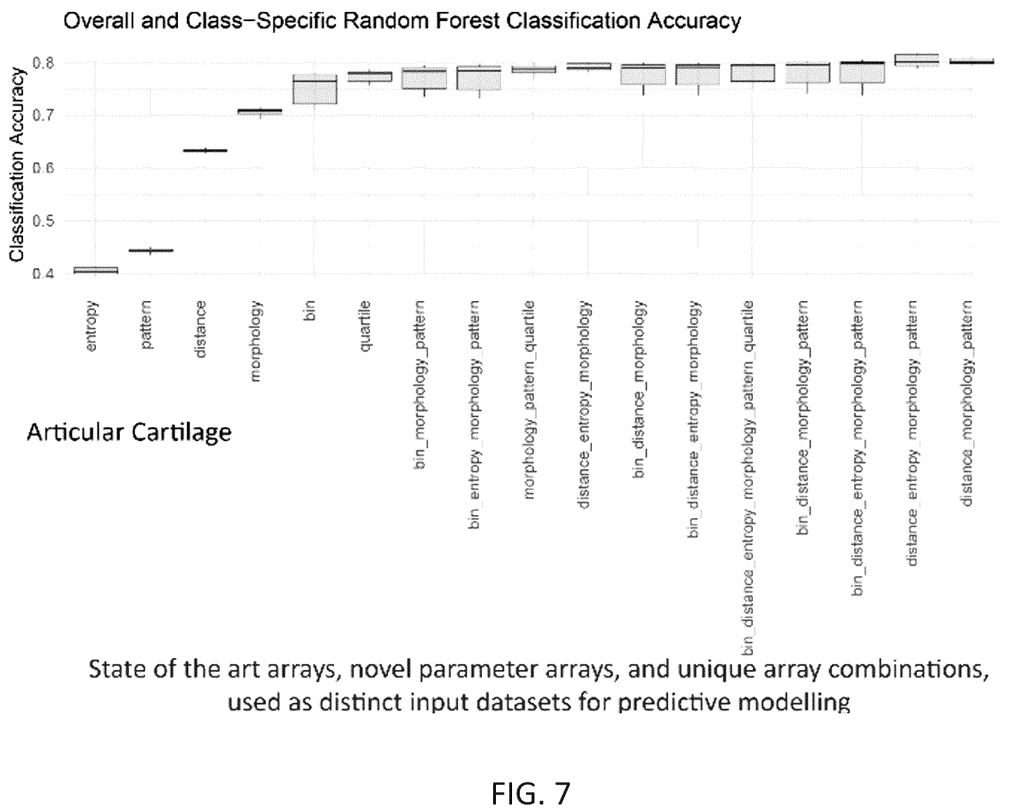

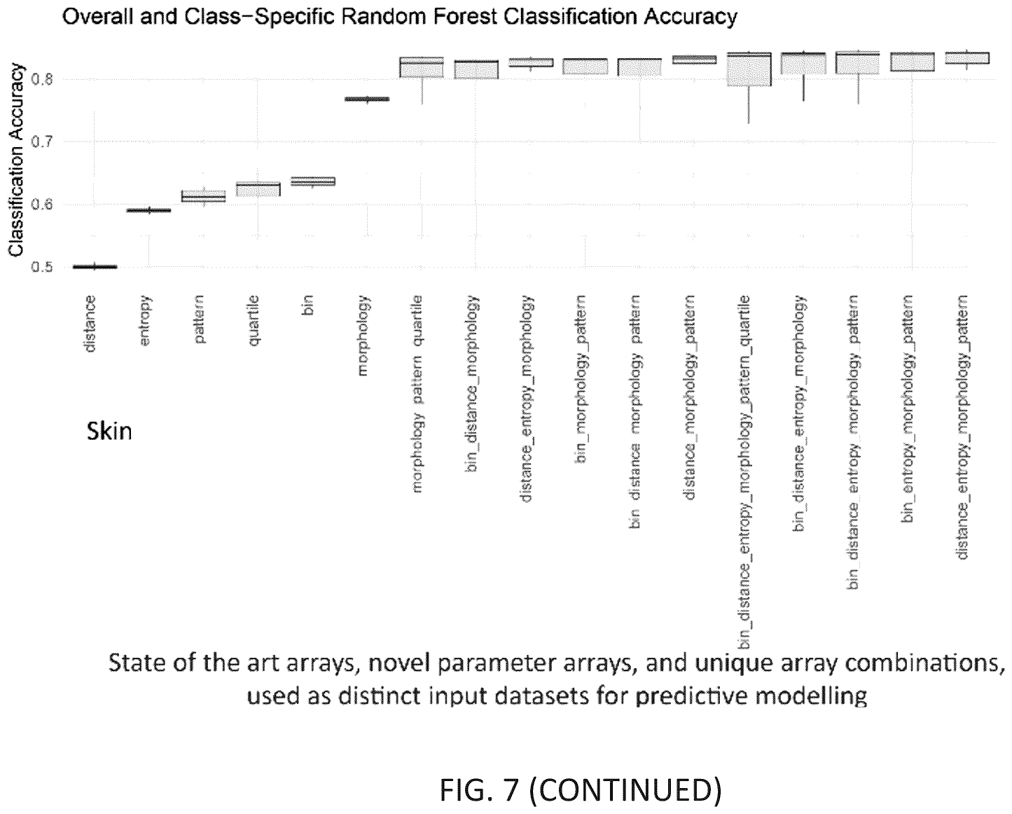

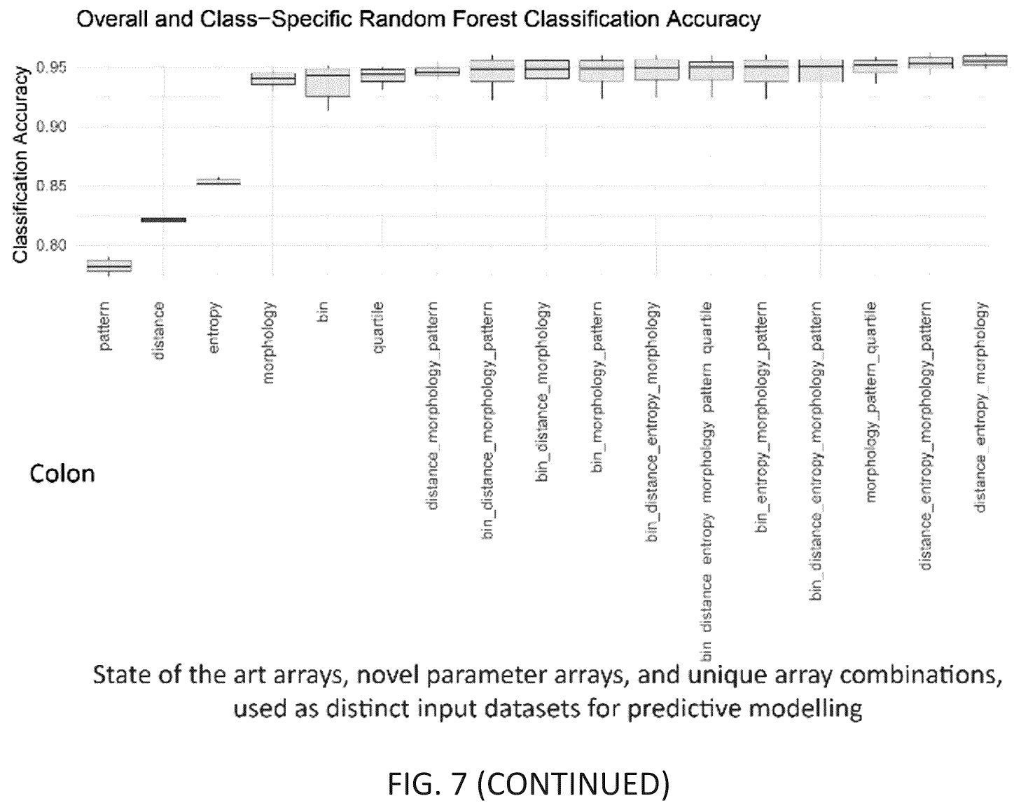

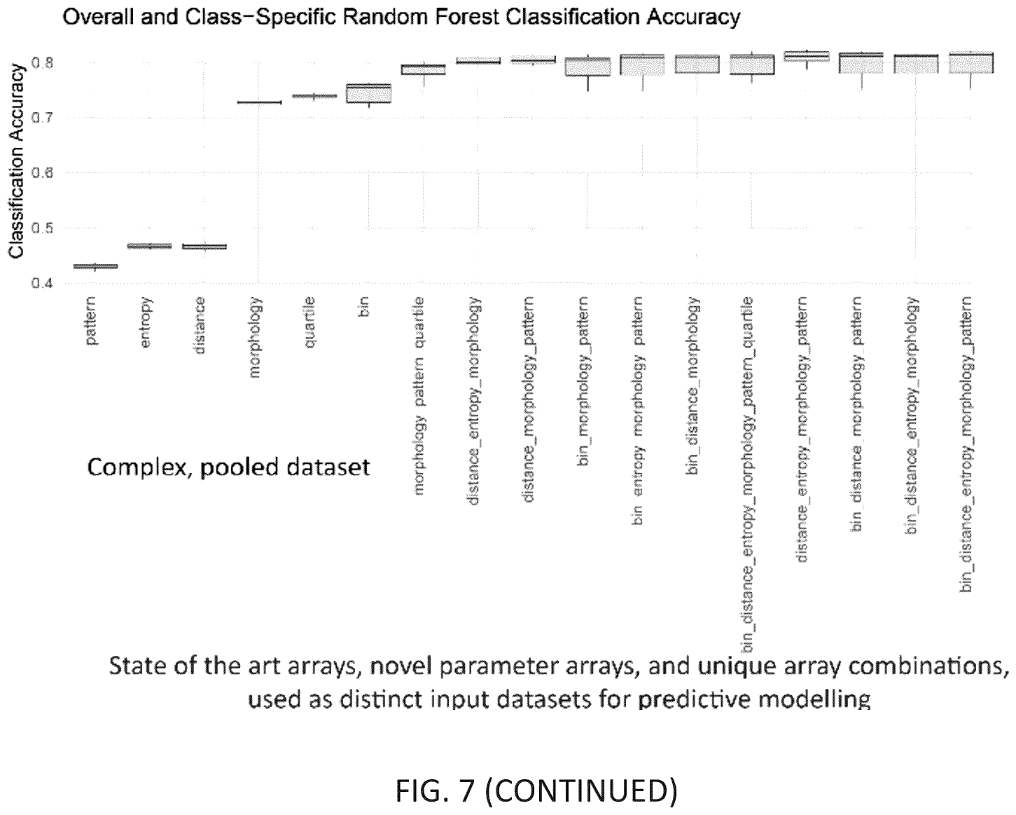

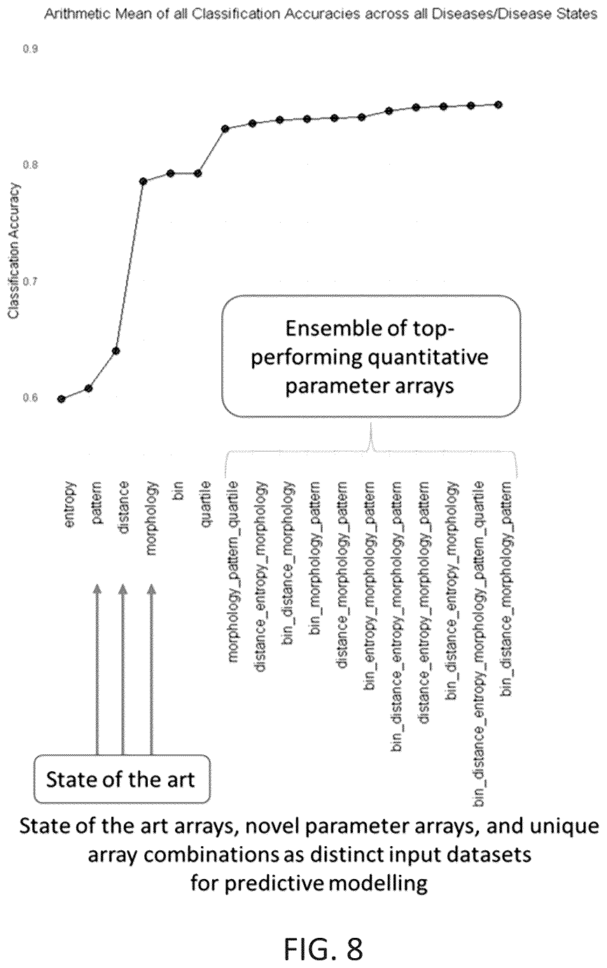

Next, the unique array combinations that consistently exhibited top-tier performance across all tissues, disease states, and the aggregated complex dataset were identified. This was achieved by sorting each classification accuracy figure (FIGS. 3-6) by increasing box plot median accuracy and, for each represented disease state, by increasing line plot accuracy. Each sorting iteration recorded the resulting order of unique array combinations, which captured their (ascending) performance as predictive modeling input for a specific disease state (data not shown). This method led to identification of the top 11 unique array combinations that consistently achieved the highest classification of accuracies in all analyzed tissues/disease states. FIG. 7 is a graph showing state-of-the-art and novel single arrays and top unique array combinations that consistently achieved the highest classification accuracies in all analyzed tissues/disease states. Shown are the classification accuracies of the three state-of-the-art arrays (pattern, distance, morphology) and their combined array (pattern_distance_morphology) vs. the top 11 unique array combinations in their respective tissues/disease states. FIG. 8 is a graph showing the feature-fused ensemble of top-performing unique array combinations and their average disease state classification accuracy performance across all tissues/disease states relative to state-of-the-art parameters. Shown are state-of-the-art parameters, novel arrays, and the feature-fused ensemble of top-performing quantitative parameter arrays, which ‘translates’ distinct facets of tissue architecture in health and changes of tissue architecture in disease(s) into comprehensive mathematical parameters as predictive modeling input data for classifying disease states, significantly surpassing the accuracy of state-of-the-art parameters.

In the above-described embodiments, the array values were calculated using threshold-segmented images of macroscopically intact and degenerating human articular cartilage tissues that depict a range of cells beneath the tissue surfaces as well as images of tissue sections of biopsies of the human skin and colon that depict a range of cells within the tissues.

The articular cartilage images depicting Calcein AM-stained cells were classified according to how the cells were arranged in spatial patterns, e.g., in strings, double strings, small and big clusters, and in a diffuse arrangement without discernible spatial patterns, according to publication [1]. These spatial patterns indicate a structural [2] and functional pathology [8, 10]. Please note that the content of these publications quantifies in part the state-of-the-art parameters distance and/or pattern. This is not in conflict with this application because the here-introduced novel arrays and the feature-fused ensemble of top-performing quantitative parameter arrays are not mentioned.

The skin images depicting Hematoxylin and Eosin-stained tissue sections were diagnosed by a professional pathologist as actinic keratosis with the histological grade KIN (keratinocyte intra-epidermal neoplasia) I, II, or III, or as moderately differentiated squamous cell carcinoma (SCC G2) [11]. Actinic keratosis are the most frequent pre-malignant lesions in the human race, whereas the SCC represents approximately 20% of non-melanoma skin cancers and is the second most prevalent type after basal cell carcinoma. Other images depicted tissue sections that were diagnosed by a professional pathologist as Morbus Bowen, which is an early form of squamous cell carcinoma that is limited to the outermost skin layer (epidermis).

The colon images depicting tissue sections of biopsies were diagnosed by a professional pathologist either as colonic adenomas (a colon tumor of benign nature, which is considered precancerous) or as colonic carcinoma (a malignant colon tumor). Note, the colon carcinoma 5-year survival rate is 91%, if it is diagnosed at a localized stage, 72%, if the cancer has spread to surrounding tissues/organs/regional lymph nodes, and 13% if the cancer has spread to distant parts of the body. These numbers illustrate the need for early diagnosis and localization, which is usually done by taking a biopsy for histopathological examination using some of the state-of-the-art parameters that are described above.

The following are some specific examples of using the described biomarker array for AI/ML learning-supported classification/diagnosis.

### Specific Example I (Skin Cancer)

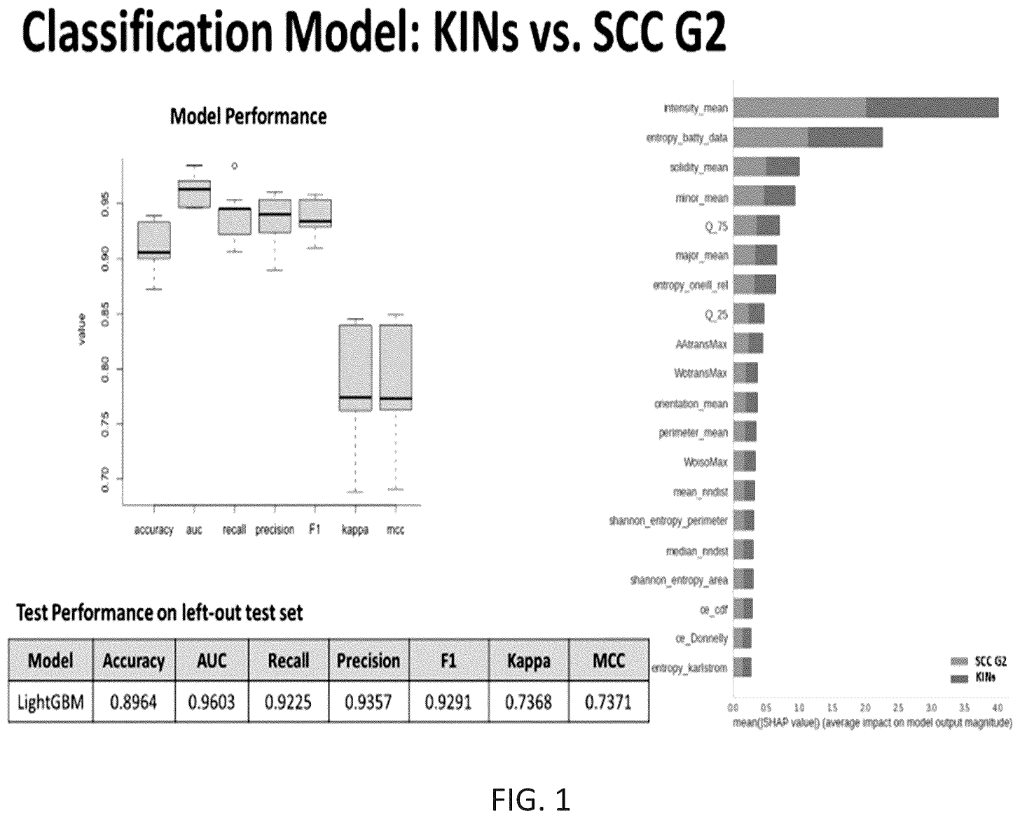

Actinic keratosis (KIN I-III), also called solar keratosis (sun-damaged skin), is a pre-cancerous area of thick, scaly, or crusty skin and is considered a precursor of squamous cell carcinoma (SCC) and may progress to SCC over time. SCC is the second most common form of skin cancer that typically develops from chronic sun-exposed areas of the body. Here, we used scans from images of pathological sections for analysis. The universal biomarker array for differentiating KIN I-III vs. SCC led to an accuracy of 89.64% correct diagnosis, using the random forest classification model and images that were diagnosed by professional pathologists. See FIG. 1—Random forest classification using the universal biomarker array for diagnosing KINs vs. SCC, in which the upper left and bottom panels give multiple parameters of model performance, and the right panel gives the SHAP (SHapley Additive explanations) values, which give the impact of each array parameter on the prediction. The test images were not included in the training data set. Note that these results were achieved with a relatively low number of images in each category (n<100), indicating that higher numbers of curated and classified images would increase the accuracy.

### Specific Example II (Colon Cancer)

Colon adenoma is a type of polyp. Up to 10% of colon adenomas can turn into cancer and is, thus, a precursor lesion of the colorectal adenocarcinoma (colon cancer). Here, we used scans from images of pathological sections for analysis.

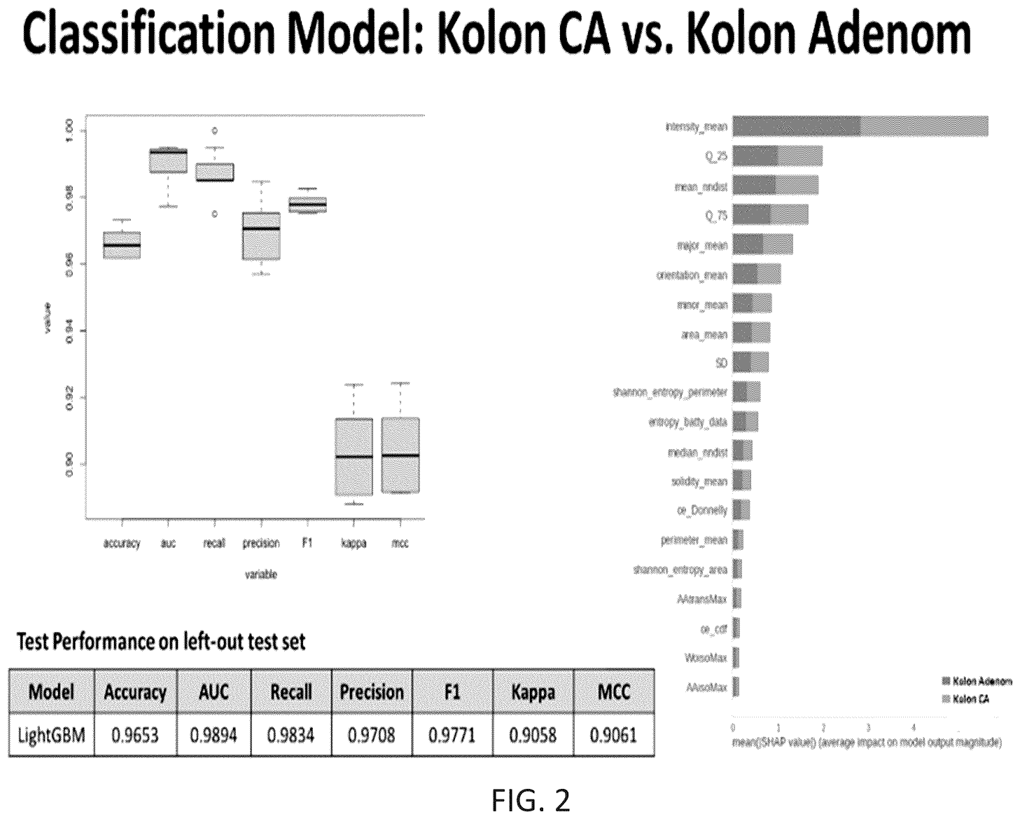

The universal biomarker array for differentiating colon adenoma vs. colon cancer led to an accuracy of 96.53% correct diagnosis, using the LightGBM classification model and images that were diagnosed by professional pathologists. See FIG. 2—Predictive classification using the universal biomarker array for diagnosing colon adenoma vs. cancer, in which the upper left and bottom panels give multiple parameters of model performance, and the right panel gives the SHAP (SHapley Additive explanations) values, which give the impact of each array parameter on the prediction. The test images were not included in the training data set. Note that these results were achieved with a relatively low number of images in each category (n<100), indicating that higher numbers of curated and classified images would increase the accuracy.

### Specific Example III (Cartilage Degeneration)

Cartilage degeneration in osteoarthritis can be recognized by the spatial organization of superficial chondrocytes (SCSO), which is a coined term and is a surrogate marker for loss of tissue functionality such as loss of nanoscale stiffness. Thus, identifying specific stages of the SCSO and answering whether a given SCSO is typical for healthy articular cartilage allows classifying healthy vs. early disease (cartilage degeneration). Here, we generated images of tissue biopsies/explants using fluorescent microscopy for analysis.

The universal biomarker array for differentiating SCSO [healthy] vs. SCSO [diseased] led to an accuracy of 91.15% correct diagnosis, using the random forest classification model and diagnosed images (see FIG. 1). The test images were not included in the training data set. Note that these results were achieved with a relatively low number of images in each category (n<100), indicating that higher numbers of curated and classified images would increase the accuracy.

The following is a description of some potential future applications envisioned by the inventor:
Use the universal biomarker array and associated methodology to develop a clinically applicable, commercially usable tool that supports medical decision making in diagnosis and treatments during medical procedures, especially of early stage diseases that so far cannot be detected or are difficult to be detected. This tool is intended to deliver real-time and user-friendly feedback to enable surgeons, internal medicine specialists and other specialists during medical procedures to diagnose early disease onset earlier than it is currently clinically possible and to localize early disease processes in the tissue(s), e.g., in combination with endo-microscopy or other suitable imaging modalities during medical procedures,
another exemplary application of this tool is the localization of early/advanced disease processes during medical procedures in the tissue for aiding guided biopsy, assessing/ensuring tumor resection, tissue debridement and implantation of biomaterials/ATMPs/RMATs (FDA, EMA) for regenerative and other purposes, e.g.,—as above—in combination with endo-microscopy or other suitable imaging modalities during medical procedures,
use the universal biomarker array and associated methodology to develop a fully clinically applicable, commercially usable tool that supports medical decision-making in diagnosis and staging that is intended for pathologists and other specialists that evaluate medical images of organs/tissues/tissue sections. An example is the realization through an imaging device and a designated software interface that streams/transmits images to us for subsequent analysis and testing and delivers an AI-supported diagnosis and staging recommendation to the pathologist or other specialists. Examples include but are not limited to diseases whose diagnoses require a biopsy, e.g., the diseases listed in this document,
use the universal biomarker array and associated methodology in conjunction with lymph node/bone marrow biopsies, peripheral blood smear, bronchoalveolar lavage, liquor puncture, brain biopsy, and other biopsy types, for cell profiling and the here described analysis,
use the universal biomarker array and associated methodology in in vitro studies, animal studies and clinical trials to generate a database with quantitative organ-, tissue-, and/or tissue section-specific and also healthy and disease state-specific information that is used as reference data for statistical and/or AI/ML supported testing of images/data of interest against database-based reference data, in order to allow the above stated applications. In this context, the database is a quantitative atlas/digital fingerprint of organ/tissue/tissue section architecture in health and disease,
use the universal biomarker array and associated methodology in conjunction with molecular imaging (e.g., via endo-microscopy but also PET, SPECT, MRI, optical imaging, ultrasound imaging, photoacoustic imaging, CT, and other imaging modalities) with markers of interest (e.g., antibodies, fragment antibodies, small molecules and other sensors or substances, including commercially available and/or, clinically applicable markers) to associate molecular imaging with early diagnosis, staging, monitoring, and disease localization in organs, tissues/tissue sections generated by the here described method,
use the universal biomarker array and associated methodology on molecular imaging signals/data, e.g., by analyzing markers of interest that were recorded by molecular imaging (e.g., antibodies, fragment antibodies, small molecules and other sensors or substances, including commercially available and/or, clinically applicable markers), to generate quantitative data usable as AI/ML input/for statistical analyses. Examples of molecular marker targets are metabolic, inflammatory, and autoimmune processes and/or anatomical and/or pathological structures,
use the above described database to relate information on disease diagnosis and localization gained through the universal biomarker array (see above) with other, in animal studies and clinical trials measured data on the molecular, sub-cellular, cellular, tissue, organ, and patient (e.g., patient-reported outcomes) level. Application examples are but are not limited to bioinformatics analyses and the testing of promising/existing/novel/to be developed drugs that target specific/early disease processes, e.g., to test existing drugs earlier in a disease, or to develop novel drugs that target previously clinically not visible disease early stages,
use the universal biomarker array and associated methodology in conjunction with ‘omics analyses, e.g., genomics, transcriptomics including spatial transcriptomics, proteomics, metabolomics, radiomics, and other fields to associate ‘omics data with early diagnosis, staging, monitoring, and disease localization in organs, tissues/tissue sections generated by the here described method, and, if applicable, use the use the universal biomarker array and associated methodology on these signals/data,
use the universal biomarker array and associated methodology in conjunction with other experimental data of interest, e.g., another particular readout of a given in vitro or ex vivo/in vivo experiments, to determine their statistical relation. Use this relationship to predict/determine experimental data of interest by using the universal biomarker array parameters or a subset of parameters as surrogate marker. An example is to predict/determine the OARSI score or the extent of surface damage of articular cartilage from the parameters or a subset of parameters (after appropriate analyses established statistical relations),
use the universal biomarker array and associated methodology on in vitro cell, organ, tissue cultures as quantitative markers and predictors of cell, organ, tissue culture performance, e.g., cell phenotype, differentiation, and other functions,
use the universal biomarker array and associated methodology on all mammalian species/issues/cells, e.g., human volunteers, human patients, and animals such as dogs, horses, and camels for the above listed purposes.
Examples of diseases for the above listed applications include but are not limited to
   Cancers and precancerous lesions, e.g., of the
      skin,
      gastrointestinal,
      lung,
      kidney,
      breast,
      liver,
      esophagus,
      brain,
      prostate,
      pancreas,
      glioblastoma,
      metastases,
   orthopedic conditions/diseases inclusive but not limited to
      injury of cartilage and other joint structures,
      degeneration of cartilage and other joint structures,
      genetic disorders involving cartilage,
      osteochondritis dissecans,
      inflammatory arthropathies (rheumatoid arthritis, juvenile idiopathic arthritis, gout, systemic lupus erythematosus, and seronegative spondyloarthropathies),
      osteoarthritis,
      post-traumatic osteoarthritis,
      chondrodysplasia
   vascular diseases, e.g.,
      atherosclerosis,
      stenosis,
      high blood pressure,
      stroke
   heart diseases, e.g.,
      coronary artery disease/atherosclerosis,
      cardiomyopathy,
      myocarditis,
      heart transplantation rejection,
   gastrointestinal diseases, e.g.
      polyps,
      gastritis/ulcus ventriculi,
      unclear gastrointestinal mass/lesion,
      cancer,
   kidney diseases, e.g.
      chronic kidney disease,
      glomerulonephritis,
      kidney fibrosis,
   liver diseases e.g.
      cirrhosis,
   inflammatory/autoinflammatory/autoimmune diseases,
   eye diseases, e.g.
      glaucoma,
      macular degeneration,
   neurodegenerative diseases, e.g.
      Parkinson's disease,
      Alzheimer's disease,
      Huntington's disease,
      amyotrophic lateral sclerosis (ALS),
      motor neuron disease,
   chronic inflammatory brain diseases, e.g.
      multiple sclerosis,
      acute demyelination,
   angiopathic diseases,
      e.g., cerebral amyloid angiopathy (CAA)
   peripheral nerve damage
   rare diseases,
   metabolic disorders/diseases, e.g.,
      diabetes

The following is information on previous publications (reference numbers in brackets refer to reference citations listed below). It should be noted that all of these studies referred only to chondrocytes/cartilage/osteoarthritis (OA). In studies from 2008 to 2014, we used the term “spatial organization” and referred only to chondrocytes/cartilage/osteoarthritis (OA). These analyses used a very limited amount of quantitative data, e.g., with 1-2 markers (Clark Evans Index, nearest neighbor distance) that are part on the here described universal biomarker array. Since 2016, we used the term “superficial zone chondrocyte spatial organization (SCSO).” A small subset of quantitative parameters was used in a few later studies on articular cartilage. Some of the terms used, relevant measurements, and key statements in relation to disease are highlighted in bold, to illustrate the very limited use of a small subgroup of the universal biomarker array and the limited context (only cartilage, chondrocytes, degeneration, osteoarthritis). It should be noted that certain cell morphology parameters have been used to describe quantitative effects of stimuli but not in conjunction with diagnosis etc.
Distinct horizontal patterns in the spatial organization of superficial zone chondrocytes of human joints [1]:

A better understanding of the unique cellular and functional properties of the superficial zone of articular cartilage may aid current strategies in tissue engineering which attempts a layered design for the repair of cartilage lesions to avert or postpone the onset of osteoarthritis. However, data pertaining to the cellular organization of non-degenerated superficial zone of articular cartilage is not available for most human joints. The present study analyzed the arrangement of chondrocytes of non-degenerated human joints (shoulder, elbow, knee, and ankle) by using fluorescence microscopy of the superficial zone in a top-down view. The resulting horizontal chondrocyte arrangements were tested for randomness, homogeneity or a significant grouping via point pattern analysis and were correlated with the joint type in which they occurred. The present study demonstrated that human superficial chondrocytes occurred in four distinct patterns of strings, clusters, pairs or single chondrocytes. Those patterns represented a significant grouping (p<0.0001) with horizontal alignment. Each articular joint surface was dominated by only one of these four patterns (p<0.001). Specific patterns correlated with specific diarthrodial joint types (p<0.001). Further studies need to establish whether these organizational patterns are a consequence of their surrounding environment or whether they are linked to a functional purpose.
Proliferative remodeling of the spatial organization of human superficial chondrocytes distant from focal early osteoarthritis [2]:

OBJECTIVE: Human superficial chondrocytes show distinct spatial organizations, and they commonly aggregate near osteoarthritic (OA) fissures. The aim of this study was to determine whether remodeling or destruction of the spatial chondrocyte organization might occur at a distance from focal (early) lesions in patients with OA. METHODS: Samples of intact cartilage (condyles, patellofemoral groove, and proximal tibia) lying distant from focal lesions of OA in grade 2 joints were compared with location-matched nondegenerative (grade 0-1) cartilage samples. Chondrocyte nuclei were stained with propidium iodide, examined by fluorescence microscopy, and the findings were recorded in a top-down view. Chondrocyte arrangements were tested for randomness or significant grouping via point pattern analyses (Clark and Evans Aggregation Index) and were correlated with the OA grade and the surface cell densities. RESULTS: In grade 2 cartilage samples, superficial chondrocytes were situated in horizontal patterns, such as strings, clusters, pairs, and singles, comparable to the patterns in nondegenerative cartilage. In intact cartilage samples from grade 2 joints, the spatial organization included a novel pattern, consisting of chondrocytes that were aligned in 2 parallel lines, building double strings. These double strings correlated significantly with an increased number of chondrocytes per group and an increased corresponding superficial zone cell density.

They were observed in all grade 2 condyles and some grade 2 tibiae, but never in grade 0-1 cartilage. CONCLUSION: This study is the first to identify a distinct spatial reorganization of human superficial chondrocytes in response to distant early OA lesions, suggesting that proliferation had occurred distant from focal early OA lesions. This spatial reorganization may serve to recruit metabolically active units as an attempt to repair focal damage.
Onset of preclinical osteoarthritis: the angular spatial organization permits early diagnosis [3]:

OBJECTIVE: Superficial articular chondrocytes display distinct spatial remodeling processes in response to the onset of distant osteoarthritis (OA). Such processes may be used to diagnose early events before manifest OA results in tissue destruction and clinical symptoms. Using a novel method of spatial quantification by calculating the angles between a chondrocyte and its surrounding neighbors, we compared maturational and degenerative changes of the cellular organizations in rat and human cartilage specimens. METHODS: The nuclei of superficial chondrocytes obtained from intact rat cartilage and from human knee cartilage, as well as from cartilage with focal and severe OA, were digitally recorded in top-down views. Their Cartesian coordinates were used to determine the nearest neighbor for each chondrocyte and the angle between these 2 cells and a reference. These angles, cellularity, nearest neighbor distances, and aggregation were analyzed as a function of location and OA severity. RESULTS: Neighboring rat chondrocytes exhibited intricate angular patterns with 4 dominant angles that were maintained during maturation and during the onset and progression of OA. Within intact cartilage, human chondrocytes demonstrated 1 dominant angle and, thus, a significantly different angular organization. With early OA onset, human chondrocytes that were located within intact cartilage displayed an increased occurrence of 4 angles; the resulting angular patterns were indistinguishable from those observed in rats. The angular remodeling was associated with location- and OA severity-dependent changes in cellularity and aggregation. CONCLUSION: This study is the first to identify the presence of angular characteristics of spatial chondrocyte organization and species-specific remodeling processes correlating with OA onset. The appearance of distinct angular and spatial patterns between neighboring chondrocytes can identify the onset of distant OA prior to microscopically visible tissue damage and possibly before clinical onset. With further development, this novel concept may become suitable for the diagnosis and followup of patients susceptible to OA.
Modeling chondrocyte patterns by elliptical cluster processes [4]:

Superficial zone chondrocytes (CHs) of human joints are spatially organized in distinct horizontal patterns. Among other factors, the type of spatial CH organization within a given articular surface depends on whether the cartilage has been derived from an intact joint or the joint is affected by osteoarthritis (OA). Furthermore, specific variations of the type of spatial organization are associated with particular states of OA. This association may prove relevant for early disease recognition based on a quantitative structural characterization of CH patterns. Therefore, we present a point process model describing the distinct morphology of CH patterns within the articular surface of intact human cartilage. This reference model for intact CH organization can be seen as a first step towards a model-based statistical diagnostic tool. Model parameters are fitted to fluorescence microscopy data by a novel statistical methodology utilizing tools from cluster and principal component analysis. This way, the complex morphology of surface CH patterns is represented by a relatively small number of model parameters. We validate the point process model by comparing biologically relevant structural characteristics between the fitted model and data derived from photomicrographs of the human articular surface using techniques from spatial statistics.
The spatial organization of joint surface chondrocytes: review of its potential roles in tissue functioning, disease and early, preclinical diagnosis of osteoarthritis [5]:

Chondrocytes display within the articular cartilage depth-dependent variations of their many properties that are comparable to the depth-dependent changes of the properties of the surrounding extracellular matrix. However, not much is known about the spatial organization of the chondrocytes throughout the tissue. Recent studies revealed that human chondrocytes display distinct spatial patterns of organization within the articular surface, and each joint surface is dominated in a typical way by one of four basic spatial patterns. The resulting complex spatial organizations correlate with the specific diarthrodial joint type, suggesting an association of the chondrocyte organization within the joint surface with the occurring biomechanical forces. In response to focal osteoarthritis (OA), the superficial chondrocytes experience a destruction of their spatial organization within the OA lesion, but they also undergo a defined remodelling process distant from the OA lesion in the remaining, intact cartilage surface. One of the biological insights that can be derived from this spatial remodelling process is that the chondrocytes are able to respond in a generalized and coordinated fashion to distant focal OA. The spatial characteristics of this process are tremendously different from the cellular aggregations typical for OA lesions, suggesting differences in the underlying mechanisms. Here we summarize the available information on the spatial organization of chondrocytes and its potential roles in cartilage functioning. The spatial organization could be used to diagnose early OA onset before manifest OA results in tissue destruction and clinical symptoms. With further development, this concept may become clinically suitable for the diagnosis of preclinical OA.
(Conference abstract) THE SPATIAL ORGANIZATION AND THE PERICELLULAR MATRIX OF HUMAN FOETAL CHONDROCYTES ARE NOT INBORN BUT INSTEAD ACQUIRED CHARACTERISTICS [6]:

Introduction In adult articular cartilage, the pericellular matrix mediates the biomechanical, biophysical and biomechanical interactions between the chondrocyte and the extracellular matrix. The PCM is also associated with the spatial organization of human superficial chondrocytes, which are situated in four distinct patterns of strings, clusters, pairs or single cells. However, little is known about the PCM and the spatial organization during fetal development. In this study, we asked the question whether fetal chondrocytes display a spatial organization comparable to that of adult chondrocytes, and whether a PCM is present or absent in the early stages of fetal cartilage development. Methods Articular cartilage sections (100 μm thickness) were prepared from the condyles of human fetal knee joints (pregnancy weeks 7-10) and from macroscopically intact condylar areas (knee joint replacement procedures). The samples were characterized by immunofluorescence microscopy and multiphoton-induced autofluorescence imaging combined with quantitative SHG signal profiling, a technique that allows quantifying the fibrillar collagen content. The spatial organization was analyzed by point pattern analyses as we described previously. For these analyses the Cartesian coordinates of each nucleus were determined by converting the immunofluorescence images into gray-scale images and finding the local gray-scale maxima with ImageJ (NIH). Results In adult superficial cartilage, the PCM surrounded single or groups of chondrocytes and defined their spatial organization of groups of cellular strings. PCM was characterized by a strong collagen VI staining signal and high collagen intensity as measured by SHG (156.7±12.4). In fetal cartilage, the condyles were characterized by a high cellular density, no recognizable spatial organization, and little amounts of extracellular matrix. No collagen VI was detected in the matrix surrounding the fetal chondrocytes. Furthermore, SHG imaging revealed that the fibrillar collagen intensity was significantly weaker (4.7±0.8; p<0.001) when compared to the adult tissues. During maturation from the fetal to the adult stage the cell density per volume decreased significantly (p<0.001). The cell distance from each fetal chondrocyte to its nearest neighbor was 15.70±0.12 μm and significantly larger for adult cartilage (35.86±1.37 μm; p<0.001). The level of spatial clustering as measured by the integral of the pair correlation function was significantly higher in the adult cartilage (p<0.001). Overall, a cellular grouping comparable to that of adult chondrocytes was not present in fetal cartilage. Together these parameters suggest that the spatial organization typical for adult condylar cartilage was not present in human fetal condylar cartilage. Discussion/Conclusion In human fetal articular cartilage the spatial organization typical for adult chondrocytes was not present. Instead, the chondrocytes were situated densely and in close proximity. This study determined for the first time that the collagenous components that are typical for the adult PCM were not present in fetal cartilage. In conclusion, the PCM and also the spatial organization of superficial human chondrocytes develop with cartilage maturation and thus are not inborn but instead acquired characteristics. Summary In adult articular cartilage, the pericellular matrix (PCM) mediates chondrocyte-matrix-interactions and is associated with the spatial cellular organization. Immunofluorescence microscopy, multiphoton-induced autofluorescence and second harmonic generation (SHG) imaging, as well as point pattern analyses revealed that both PCM and spatial organization were absent in fetal chondrocytes.
Loss of spatial organization and destruction of the pericellular matrix in early osteoarthritis in vivo and in a novel in vitro methodology [7]:

OBJECTIVES: Current repair procedures for articular cartilage (AC) cannot restore the tissue's original form and function because neither changes in its architectural blueprint throughout life nor the respective biological understanding is fully available. We asked whether two unique elements of human cartilage architecture, the chondrocyte-surrounding pericellular matrix (PCM) and the superficial chondrocyte spatial organization (SCSO) beneath the articular surface (AS) are congenital, stable or dynamic throughout life. We hypothesized that inducing chondrocyte proliferation in vitro impairs organization and PCM and induces an advanced osteoarthritis (OA)-like structural phenotype of human cartilage. METHODS: We recorded propidium-iodine-stained fetal and adult cartilage explants, arranged stages of organization into a sequence, and created a lifetime-summarizing SCSO model. To replicate the OA-associated dynamics revealed by our model, and to test our hypothesis, we transduced specifically early OA-explants with hFGF-2 for inducing proliferation. The PCM was examined using immuno- and auto-fluorescence, multiphoton second-harmonic-generation (SHG), and scanning electron microscopy (SEM). RESULTS: Spatial organization evolved from fetal homogeneity, peaked with adult string-like arrangements, but was completely lost in OA. Loss of organization included PCM perforation (local micro-fibrillar collagen intensity decrease) and destruction [regional collagen type VI (CollVI) signal weakness or absence]. Importantly, both loss of organization and PCM destruction were successfully recapitulated in FGF-2-transduced explants. CONCLUSION: Induced proliferation of spatially characterized early OA-chondrocytes within standardized explants recapitulated the full range of loss of SCSO and PCM destruction, introducing a novel in vitro methodology. This methodology induces a structural phenotype of human cartilage that is similar to advanced OA and potentially of significance and utility.
Proof-of-concept for the detection of early osteoarthritis pathology by clinically applicable endo-microscopy and quantitative AI-supported optical biopsy [8]:

OBJECTIVE: Clinical trials for osteoarthritis (OA), the leading cause of global disability, are unable to pinpoint the early, potentially reversible disease with clinical technology. Hence, disease-modifying drug candidates cannot be tested early in the disease. To overcome this obstacle, we asked whether early OA-pathology detection is possible with current clinical technology. METHODS: We determined the relationship between two sensitive early OA markers, atomic force microscopy (AFM)-measured human articular cartilage (AC) surface stiffness, and location-matched superficial zone chondrocyte spatial organizations (SCSOs), asking whether a significant loss of surface stiffness can be detected in early OA SCSO stages. We then tested whether current clinical technology can visualize and accurately diagnose the SCSOs using an approved probe-based confocal laser-endomicroscope and a random forest (RF) model. RESULTS: We demonstrated a correlation between AC surface stiffness and the SCSO (r(rm)=−0.91; 95% CI: −0.97, −0.73), and an extensive loss of surface stiffness specifically in those ACs with early OA-typical SCSO (95% CIs: string SCSO: 269-173 kPa, double string SCSO: 77-46 kPa). This established the SCSO as a visualizable, functionally relevant surrogate marker of early OA AC surface pathology. Moreover, SCSO-based stiffness discrimination worked well in each patient's AC. We then demonstrated feasibility of visualizing the SCSO by clinical laser-endo-microscopy and, importantly, accurate SCSO diagnosis using RF. CONCLUSION: We present the proof-of-concept of early OA-pathology detection with available clinical technology, introducing a future-oriented, AI-supported, non-destructive quantitative optical biopsy for early disease detection. Operationalizing SCSO recognition, this approach allows testing for correlations between local tissue architectures with other experimental and clinical read-outs, but needs clinical validation and a larger sample size for defining diagnostic thresholds.
Hybrid fluorescence-AFM explores articular surface degeneration in early osteoarthritis across length scales [9]:

Atomic force microscopy (AFM) has become a powerful tool for the characterization of materials at the nanoscale. Nevertheless, its application to hierarchical biological tissue like cartilage is still limited. One reason is that such samples are usually millimeters in size, while the AFM delivers much more localized information. Here a combination of AFM and fluorescence microscopy is presented where features on a millimeter sized tissue sample are selected by fluorescence microscopy on the micrometer scale and then mapped down to nanometer precision by AFM under native conditions. This served us to show that local changes in the organization of fluorescent stained cells, a marker for early osteoarthritis, correlate with a significant local reduction of the elastic modulus, local thinning of the collagen fibers, and a roughening of the articular surface. This approach is not only relevant for cartilage, but in general for the characterization of native biological tissue from the macro-to the nanoscale. STATEMENT OF SIGNIFICANCE: Different length scales have to be studied to understand the function and dysfunction of hierarchically organized biomaterials or tissues. Here we combine a highly stable AFM with fluorescence microscopy and precisely motorized movement to correlate micro- and nanoscopic properties of articular cartilage on a millimeter sized sample under native conditions. This is necessary for unraveling the relationship between microscale organization of chondrocytes, micrometer scale changes in articular cartilage properties and nanoscale organization of collagen (including D-banding). We anticipate that such studies pave the way for a guided design of hierarchical biomaterials.
The articular cartilage surface is impaired by a loss of thick collagen fibers and formation of type I collagen in early osteoarthritis [10]:

Osteoarthritis (OA) is a joint disease affecting millions of patients worldwide. During OA onset and progression, the articular cartilage is destroyed, but the underlying complex mechanisms remain unclear. Here, we uncover changes in the thickness of collagen fibers and their composition at the onset of OA. For articular cartilage explants from knee joints of OA patients, we find that type I collagen-rich fibrocartilage-like tissue was formed in macroscopically intact cartilage, distant from OA lesions. Importantly, the number of thick fibers (>100 nm) has decreased early in the disease, followed by complete absence of thick fibers in advanced OA. We have obtained these results by a combination of high-resolution atomic force microscopy imaging under near-native conditions, immunofluorescence, scanning electron microscopy and a fluorescence-based classification of the superficial chondrocyte spatial organization. Taken together, our data suggests that the loss of tissue functionality in early OA cartilage is caused by a reduction of thick type II collagen fibers, likely due to the formation of type I collagen-rich fibrocartilage, followed by the development of focal defects in later OA stages. We anticipate that such an integrative characterization will be very beneficial for an in-depth understanding of other native biological tissues and the development of sustainable biomaterials. STATEMENT OF SIGNIFICANCE: In early osteoarthritis (OA) the cartilage appears macroscopically intact. However, this study demonstrates that the collagen network already changes in early OA by collagen fiber thinning and the formation of fibrocartilage-like tissue. Both nanoscopic deficiencies already occur in macroscopically intact regions of the human knee joint and are likely connected to processes that result in a weakened extracellular matrix. This study enhances the understanding of earliest progressive cartilage degeneration in the absence of external damage. The results suggest a determination of the mean collagen fiber thickness as a new target for the detection of early OA and a regulation of type I collagen synthesis as a new path for OA treatment.

The following references citations are used in the discussion above.
[1] Rolauffs, B., et al., Distinct horizontal patterns in the spatial organization of superficial zone chondrocytes of human joints. Journal of Structural Biology, 2008. 162(2): p. 335-344.
[2] Rolauffs, B., et al., Proliferative remodeling of the spatial organization of human superficial chondrocytes distant from focal early osteoarthritis. Arthritis Rheum, 2010. 62(2): p. 489-98.
[3] Rolauffs, B., et al., Onset of preclinical osteoarthritis: the angular spatial organization permits early diagnosis. Arthritis Rheum, 2011. 63(6): p. 1637-47.
[4] Meinhardt, M., et al., Modeling chondrocyte patterns by elliptical cluster processes. 2012. 177(2): p. 447-458.
[5] Aicher, W. K. and B. Rolauffs, The spatial organisation of joint surface chondrocytes: review of its potential roles in tissue functioning, disease and early, preclinical diagnosis of osteoarthritis. Ann Rheum Dis, 2014. 73(4): p. 645-53.
[6] Felka, T., et al., THE SPATIAL ORGANISATION AND THE PERICELLULAR MATRIX OF HUMAN FOETAL CHONDROCYTES ARE NOT INBORN BUT INSTEAD ACQUIRED CHARACTERISTICS. Bone & Joint Journal Orthopaedic Proceedings Supplement, 2014. 96-B(SUPP 11): p. 323.
[7] Felka, T., et al., Loss of spatial organization and destruction of the pericellular matrix in early osteoarthritis in vivo and in a novel in vitro methodology. Osteoarthritis Cartilage, 2016. 24(7): p. 1200-9.
[8] Tschaikowsky, M., et al., Proof-of-concept for the detection of early osteoarthritis pathology by clinically applicable endo-microscopy and quantitative AI-supported optical biopsy. Osteoarthritis Cartilage, 2021. 29(2): p. 269-279.
[9] Tschaikowsky, M., et al., Hybrid fluorescence-AFM explores articular surface degeneration in early osteoarthritis across length scales. Acta Biomater, 2021. 126: p. 315-325.
[10] Tschaikowsky, M., et al., The articular cartilage surface is impaired by a loss of thick collagen fibers and formation of type I collagen in early osteoarthritis. Acta Biomaterialia, 2022. 146: p. 274-283.
[11] Yantsos, V., et al., Incipient intraepidermal cutaneous squamous cell carcinoma: a proposal for reclassifying and grading solar (actinic) keratoses. Semin Cutan Med Surg 1999; 18(1):3-14. (In eng). DOI: 10.1016/s1085-5629(99)80003-0.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In alternative embodiments, the disclosed apparatus and methods (e.g., as in any flow charts or logic flows described above) may be implemented as a computer program product for use with a computer system. Such implementation may include a series of computer instructions fixed on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as a tangible, non-transitory semiconductor, magnetic, optical or other memory device, and may be transmitted using any communications technology, such as optical, infrared, RF/microwave, or other transmission technologies over any appropriate medium, e.g., wired (e.g., wire, coaxial cable, fiber optic cable, etc.) or wireless (e.g., through air or space).

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads. Software systems may be implemented using various architectures such as a monolithic architecture or a microservices architecture.

Importantly, it should be noted that embodiments of the present invention may employ conventional components such as conventional computers (e.g., off-the-shelf PCs, mainframes, microprocessors), conventional programmable logic devices (e.g., off-the shelf FPGAs or PLDs), or conventional hardware components (e.g., off-the-shelf ASICs or discrete hardware components) which, when programmed or configured to perform the non-conventional methods described herein, produce non-conventional devices or systems. Thus, there is nothing conventional about the inventions described herein because even when embodiments are implemented using conventional components, the resulting devices and systems are necessarily non-conventional because, absent special programming or configuration, the conventional components do not inherently perform the described non-conventional functions.

The activities described and claimed herein provide technological solutions to problems that arise squarely in the realm of technology. These solutions as a whole are not well-understood, routine, or conventional and in any case provide practical applications that transform and improve computers and computer routing systems.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:
P1. A method for disease diagnosis, the method comprising: receiving images of organs/tissues/tissue sections; performing automated image analysis for recognizing in the images organs/tissues/tissue sections/cells vs. image background, building regions of interest (ROIs) in non-background regions, and ROI-based segmentation (i) of cells for analyses of the segmented cells and (ii) analyses of the inverted segmentation for analyses of the space between the cells (the matrix); calculating a range of mathematical parameters on multiple levels for collective use as a universal biomarker array; storing curated (e.g., organ/tissue/tissue section/cell-and disease state-specific) diagnosed/classified/annotated universal biomarker array data in a database to produce database-based reference data; and using the images/data of interest for statistical and/or AI/ML supported testing against the database-based reference data for early diagnosis and localization of disease processes.
P2. A system comprising at least one processor and at least one memory containing instructions which, when executed by the at least one processor, causes the system to perform processes comprising: receiving images of organs/tissues/tissue sections; performing automated image analysis for recognizing in the images organs/tissues/tissue sections/cells vs. image background, building regions of interest (ROIs) in non-background regions, and ROI-based segmentation (i) of cells for analyses of the segmented cells and (ii) analyses of the inverted segmentation for analyses of the space between the cells (the matrix); calculating a range of mathematical parameters on multiple levels for collective use as a universal biomarker array; storing curated (e.g., organ/tissue/tissue section/cell-and disease state-specific) diagnosed/classified/annotated universal biomarker array data in a database to produce database-based reference data; and using the images/data of interest for statistical and/or AI/ML supported testing against the database-based reference data for early diagnosis and localization of disease processes.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. A computer-implemented method for disease diagnosis, the method comprising:
receiving digital tissue images showing tissue details at a cellular level;
performing automated image analysis on the received digital tissue images to identify cells of interest for analysis;
calculating a range of mathematical parameters on multiple levels for collective use as a universal biomarker array including a pattern array, a distance array, a morphology array, a spatial entropy array, a bin array in which absolute parameter values are translated to relative information by assigning them to specific bin positions, and a quartile array in which absolute parameter values are translated to relative information by assigning values to specific quartiles; and
analyzing the universal biomarker array relative to database-based reference data for early diagnosis and localization of disease processes.

2. (canceled)

3. The method of claim 1, wherein performing automated image processing comprises identifying cells of interest relative to an image background, building at least one region of interest (ROI) in a non-background region, and performing ROI-based segmentation (i) of cells for analyses of the segmented cells and (ii) analyses of an inverted segmentation for analyses of spaces between the cells.

4. The method of claim 1, wherein the array of spatial entropy parameters include Batty (absolute, relative), Contagion, Karlstrom (absolute, relative), O Neill (absolute, relative), and Parredw parameters.

5. The method of claim 1, wherein calculating the bin array comprises assigning each of a plurality of parameters into relative class-and range-specific bin positions across an entire range of parameter values, wherein a total number of bins is calculated by multiplying a number of disease states with a number of bins per state, optionally wherein the translation of data into relative class-and range-specific bin positions is performed on the parameters of the spatial entropy array, the pattern array. the distance array, and the morphology array.

6. (canceled)

7. (canceled)

8. The method of claim 1, wherein calculating the quartile array comprises:
assigning each of a plurality of parameter values to a disease state-specific quartile position; and
translating each of the plurality of parameter values into a disease range-specific quartile position, wherein the translation of data into relative state- and range-specific quartile positions is performed on the parameters of the spatial entropy array, the pattern array, the distance array, and the morphology array.

9. The method of claim 1, wherein analyzing the universal biomarker array relative to database-based reference data comprises:
providing the universal biomarker array to an AI/ML system trained on universal biomarker array data to detect early diagnosis and localization of disease processes, optionally wherein the AI/ML system utilizes random forest regression to detect early diagnosis and localization of disease processes.

10. (canceled)

11. A system for disease diagnosis, the system comprising:
a computer system having at least one computer processor and associated memory containing computer program instructions which, when executed by the at least one computer processor, performs computer processes comprising:
receiving digital tissue images showing tissue details at a cellular level;
performing automated image analysis on the received digital tissue images to identify cells of interest for analysis;
calculating a range of mathematical parameters on multiple levels for collective use as a universal biomarker array including a pattern array, a distance array, a morphology array, a spatial entropy array, a bin array in which absolute parameter values are translated to relative information by assigning them to specific bin positions, and a quartile array in which absolute parameter values are translated to relative information by assigning values to specific quartiles; and
analyzing the universal biomarker array relative to database-based reference data for early diagnosis and localization of disease processes.

12. (canceled)

13. The system of claim 11, wherein performing automated image processing comprises identifying cells of interest relative to an image background, building at least one region of interest (ROI) in a non-background region, and performing ROI-based segmentation (i) of cells for analyses of the segmented cells and (ii) analyses of an inverted segmentation for analyses of spaces between the cells.

14. The system of claim 11, wherein the array of spatial entropy parameters include Batty (absolute, relative), Contagion, Karlstrom (absolute, relative), O Neill (absolute, relative), and Parredw parameters.

15. The system of claim 11, wherein calculating the bin array comprises assigning each of a plurality of parameters into relative class- and range-specific bin positions across an entire range of parameter values, wherein a total number of bins is calculated by multiplying a number of disease states with a number of bins per state, optionally wherein the translation of data into relative class- and range-specific bin positions is performed on the parameters of the spatial entropy array, the pattern array, the distance array, and the morphology array.

16. (canceled)

17. (canceled)

18. The system of claim 11, wherein calculating the quartile array comprises:
assigning each of a plurality of parameter values to a disease state-specific quartile position; and
translating each of the plurality of parameter values into a disease range-specific quartile position, wherein the translation of data into relative state- and range-specific quartile positions is performed on the parameters of the spatial entropy array, the pattern array, the distance array, and the morphology array.

19. The system of claim 11, wherein analyzing the universal biomarker array relative to database-based reference data comprises:
providing the universal biomarker array to an AI/ML system trained on universal biomarker array data to detect early diagnosis and localization of disease processes, optionally wherein the AI/ML system utilizes random forest regression to detect early diagnosis and localization of disease processes.

20-30. (canceled)

31. The method of claim 1, wherein the digital tissue images include digital tissue images of cells treated with an auto-fluorescent drug including at least one of:
tetracycline;
ciprofloxacin;
chinin; or
doxycycline with divalent cations in conjunction with a surgical isotonic solution, optionally wherein the digital tissue images include digital tissue images of cells treated with the auto-fluorescent drug excited in the range of 350-400 nm, e.g., with UV-A light.

32. The system of claim 11, wherein the digital tissue images include digital tissue images of cells treated with an auto-fluorescent drug including at least one of:
tetracycline;
ciprofloxacin;
chinin; or
doxycycline with divalent cations in conjunction with a surgical isotonic solution, optionally wherein the digital tissue images include digital tissue images of cells treated with the auto-fluorescent drug excited in the range of 350-400 nm, e.g., with UV-A light.

33. A computer program product comprising at least one tangible, non-transitory computer readable medium having embodied therein computer program instructions for disease diagnosis which, when executed by at least one computer processor, performs computer processes comprising:
receiving digital tissue images showing tissue details at a cellular level;
performing automated image analysis on the received digital tissue images to identify cells of interest for analysis;
calculating a range of mathematical parameters on multiple levels for collective use as a universal biomarker array including a pattern array, a distance array, a morphology array, a spatial entropy array, a bin array in which absolute parameter values are translated to relative information by assigning them to specific bin positions, and a quartile array in which absolute parameter values are translated to relative information by assigning values to specific quartiles; and
analyzing the universal biomarker array relative to database-based reference data for early diagnosis and localization of disease processes.
